(19) United States
(12) Patent Application Publication
(10) Pub. No.: US 2025/0333775 A1
(43) Pub. Date: Oct. 30, 2025

(54) DIAGNOSTIC TEST AND THERAPY FOR PATIENTS WITH AUTO-BREWERY SYNDROME

(71) Applicant: The Regents of the University of California, Oakland, CA (US)

(72) Inventors: Bernd Schnabl, San Diego, CA (US); Cynthia L. Hsu, La Jolla, CA (US)

(21) Appl. No.: 19/190,101

(22) Filed: Apr. 25, 2025

Related U.S. Application Data
(60) Provisional application No. 63/639,358, filed on Apr. 26, 2024.

(51) Int. Cl. C12Q 1/06; A61B 10/00; A61K 31/165; A61P 1/00; C12Q 1/32; C12Q 1/6883; G01N 21/33; G01N 21/359; G01N 30/02

(52) U.S. Cl. CPC C12Q 1/06; A61B 10/0038; A61K 31/165; A61P 1/00; C12Q 1/32; C12Q 1/6883; C12Q 2600/158; G01N 21/33; G01N 21/359; G01N 2030/027; G01N 2333/245; G01N 2333/26; G01N 2333/904; G01N 2800/06

(57) ABSTRACT

Provided herein are methods of analyzing a fecal sample including measuring the amount of ethanol produced by the fecal sample and measuring the presence, absence, or relative abundance of Ruminococcus gnavus, Escherichia coli, or Klebsiella pneumoniae in the fecal sample compared to a reference sample, and/or measuring the expression of genes associated with the heterolactic fermentation pathway, the mixed acid fermentation pathway, the ethanolamine utilization pathway, and/or acetylene degradation pathway. Also provided herein are methods of treating a condition associated with gut alcohol production in a subject including analyzing a fecal sample from a subject, determining the subject has a gut alcohol production condition, and administering to the subject an effective amount of a treatment comprising a probiotic with optional resistant starch, fecal microbiota transplantation (FMT), an antimicrobial agent, a microbial enzyme inhibitor, or a combination thereof.

### CLAIM OF PRIORITY

This application claims the benefit of U.S. Provisional Application Ser. No. 63/639,358, filed on Apr. 26, 2024. The entire contents of the foregoing are incorporated herein by reference.

### FEDERALLY SPONSORED RESEARCH OR DEVELOPMENT

This invention was made with Government support under Grant No. K 99A A 031328 awarded by the National Institutes of Health. The Government has certain rights in the invention.

### TECHNICAL FIELD

The present disclosure relates to methods and materials for treating Auto-brewery Syndrome.

### BACKGROUND

Auto-brewery Syndrome (ABS), also known as gut fermentation syndrome, is a rarely diagnosed condition where patients exhibit symptoms of intoxication due to systemic absorption of pathologic levels of ethanol production by dysregulated gut microbiota [1]. Currently this condition has only been described in case reports and case-control studies [2,3], as the gold standard for diagnosis consists of a monitored rise in blood alcohol concentration while the patient is in a supervised clinical setting and is typically facilitated with administration of an oral glucose load [4]. These diagnostic methods are resource-intensive and are often not readily accessible to patients or clinicians or covered by payers. Consequently, many patients will visit multiple medical centers only to be dismissed as surreptitious drinkers and leave without a diagnosis. These patients often experience significant complications similar to those associated with alcohol use disorder, including serious family, social, and legal problems [5].

The etiology and triggers of Auto-brewery Syndrome are poorly understood. Auto-brewery Syndrome has been associated with gastrointestinal illnesses such as short gut syndrome and Crohn's disease and several early reports indicated that yeast overgrowth was the culprit, hence the name [2,6,7]. More recent case reports have identified ethanol-producing strains of Klebsiella species in patients with Auto-brewery Syndrome and liver disease [3,8]. However, existing studies have compared patient microbiomes to normal healthy controls, which do not account for inherent environmental and dietary exposures that affect the gut microbiome.

### SUMMARY

The present disclosure provides methods for treating a condition associated with gut alcohol production in a subject, the method comprising: (a) analyzing a fecal sample obtained from a subject, wherein analyzing the fecal sample comprises culturing or having cultured the fecal sample and performing or having performed metagenomic sequencing of the fecal sample; (b) determining that the subject has a gut alcohol production condition; and (c) administering to the subject an effective amount of a treatment comprising a probiotic with optional resistant starch, fecal microbiota transplantation (FMT), an antimicrobial agent, a microbial enzyme inhibitor, or a combination thereof.

In some embodiments, the fecal sample is obtained from the subject if the subject is experiencing or has experienced symptoms of a condition associated with gut alcohol production. In some embodiments, the fecal sample is obtained from the subject if the subject has experienced three or more episodes of symptoms of a condition associated with gut alcohol production within the last year.

In some embodiments, the condition associated with gut alcohol production comprises Auto Brewery Syndrome (ABS), steatotic liver disease, or alcohol use disorder. In some embodiments, the steatotic liver diseases comprise metabolic dysfunction-associated steatotic liver disease (MASLD), metabolic dysfunction and alcohol-associated liver disease (MetALD), or alcohol-associated liver disease (ALD).

In some embodiments, culturing or having cultured the fecal sample comprises culturing the fecal sample in a bioreactor.

In some embodiments, performing or having performed metagenomic sequencing of the fecal sample comprises shotgun metagenomic sequencing of the fecal sample. In some embodiments, performing or having performed metagenomic sequencing of the fecal sample is not and/or does not include 16S rRNA sequencing.

In some embodiments, determining the subject has a gut alcohol production condition comprises identifying the fecal sample as meeting one or more of the following conditions: (a) the amount of ethanol produced by the fecal sample is higher than 8.21 mg/dL in bioreactor culture; (b) the abundance of Ruminococcus gnavus, Escherichia coli, or Klebsiella pneumoniae is higher in fecal sample obtained from the subject as compared to a reference fecal sample; (c) enzymes in the heterolactic fermentation pathway, the mixed acid fermentation pathway, the ethanolamine utilization pathway, and/or acetylene degradation pathway are over-represented in the fecal sample compared to a reference fecal sample; and (d) genes associated with one or more of the enzymes identified in (c) are over-represented in the fecal sample compared to a reference fecal sample. In some embodiments, the fecal sample meets two or more of the conditions.

In some embodiments, the amount of ethanol produced by the fecal sample is determined using anaerobic bioreactor culture and high-performance liquid chromatography (HPLC).

In some embodiments, the enzymes comprise one or more of fumarate reductase, pyruvate formate lyase, phosphotransacetylase, acetaldehyde dehydrogenase, alcohol dehydrogenase (ADH), ethanolamine transporter, and ethanolamine ammonia-lyase. In some embodiments, the ADH comprises E. coli ADH.

In some embodiments, the antimicrobial agent comprises an antibacterial agent and/or an antifungal agent. In some embodiments, the antibacterial agent comprises chloramphenicol.

In some embodiments, the microbial enzyme inhibitor comprises thiabendazole, cambendazole, fenbendazole, oxfendazole, methacrylate, acryalate, metadoxine, disulfiram, fomepizole, or combinations thereof.

Also provided herein are methods for analyzing a fecal sample, the method comprising: (a) measuring the amount of ethanol produced by the fecal sample; and (b) measuring the relative abundance of Ruminococcus gnavus, Escherichia coli, or Klebsiella pneumoniae in the fecal sample compared to that in a reference sample, and/or measuring the expression of genes associated with the heterolactic fermentation pathway, the mixed acid fermentation pathway, the ethanolamine utilization pathway, and/or acetylene degradation pathway, thereby analyzing a fecal sample.

In some embodiments, measuring the amount of ethanol comprises culturing the fecal sample in a bioreactor, and determining ethanol production of the cultured fecal sample by chromatography, spectrophotometry, or enzymatic assay. In some embodiments, the chromatography is gas chromatography (GC) or high performance liquid chromatography (HPLC). In some embodiments, the spectrophotometry comprises dichromate oxidation, UV-vis spectrophotometry, near-infrared (NIR) spectrophotometry, or probe-based spectrophotometry. In some embodiments, the enzymatic assay comprises ADH assay, or fluorometric assays. In some embodiments, measuring the expression of genes comprises shotgun metagenomic sequencing.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-ID show gut microbial ethanol production in bioreactor cultures. (1A) Ethanol measured in microbiota bioreactor cultures from patients with clinically documented Auto-brewery Syndrome during a flare (yellow), during remission (pink), and from household partners (HHP; green) at 0, 6, and 24 hours. Dashed line represents detection limit by high-performance liquid chromatography. (1B) Ethanol measurements from (1A) at 24 hours. (1C) Spearman's rank correlation analysis between subjects' blood alcohol concentration at the time of stool collection and ethanol measured in bioreactor cultures at 24 hours for patients with ABS during flare and remission. (1D) Ethanol measurements from bioreactor cultures of fecal microbiota from household partners (HHP) and patients with clinically documented Auto-brewery Syndrome during remission and during a flare, treated with dimethyl sulfoxide (DMSO, vehicle), amphotericin B (AmpB), or chloramphenicol (Chloram), at 24 hours.

FIGS. 2A-2E show gut microbial taxonomic composition of patients with Auto-brewery Syndrome. (2A) Relative abundance of gut bacteria at the phyla level in patients with clinically documented Auto-brewery Syndrome in remission or during a flare or their household partners (HHP). Each column represents one patient or household partner and the relative abundance of conditions with more than one sample were averaged. (2B) Boxplot quantifying relative abundance of bacteria in the Proteobacteria phylum. (2C) Spearman's rank correlation analysis of blood alcohol concentration in patients with Auto-brewery Syndrome at the time of stool collection and the relative abundance of Proteobacteria in the corresponding sample. (2D) Alpha diversity, as measured by Inverse Simpson index, of gut microbial species in healthy controls (HC), household partners (HHP), patients with ABS in remission and in flare. (2E) Principal component analysis demonstrating beta diversity of microbial composition at the species level.

FIGS. 3A-3G provide results from metabolic pathway analysis that reveals differences in gut microbial function. (3A) Microbial metabolic pathways significantly associated with the “Flare” condition as determined by MaAsLin2 (q<0.05 and Coefficient>1.0 for Flare vs Remission; q<0.05 and Coefficient>2.5 for Flare vs HHP; qval<0.001 and Coefficient>4.0 for Flare vs Healthy Controls (H C)). Positive coefficient indicates increased abundance in ‘Flare’ compared with other disease states, while negative coefficient indicates decreased abundance. Bar color density is proportional to the significance of the association, represented by the q-value. All detected associations are adjusted for households as random effects and disease status as fixed effects. (3B-3G) Abundance of different metabolic pathways: mixed acid fermentation pathway (3B), heterolactic fermentation pathway (3C), ethanolamine utilization pathway (3D), acetylene degradation pathway (3E), demethylmenaquinol 6 biosynthesis pathway (3F), and TCA cycle (acetate producers) (3G). Grey lines connect samples within the same household.

FIGS. 4A-4B show gut microbial composition after fecal microbiota transplantation. (4A) Principal component analysis of gut microbial taxonomic composition at the species level of one patient and their household partner (HHP) before, during, and after two different treatment courses of fecal microbiota transplantation (FMT), during remission or flare. (4B) Relative abundance of gut microbiota at the phyla level, relative abundance of Escherichia coli, blood alcohol concentration, fermentation pathway enrichment, AST, and ALT of samples in (4A) across time. nm for BAC=not measured. Green arrows indicate broad spectrum antibiotics and black arrows indicate FMT capsule administration.

FIGS. 5A-5B. (5A) Flowchart of individuals screened for our study and reasons for exclusion. (5B) Timeline of biospecimen collection from each enrolled household by patients during flare or remission, or household partners.

FIG. 6. Ethanol measurements from bioreactor cultures of fecal microbiota from patients with Auto-brewery Syndrome during a flare, grown in LB broth supplemented with 2 mM, 100 mM, or 200 mM glucose, at 24 hours.

FIGS. 7A-7D. (7A) Microbial species significantly associated with the “Flare” condition (q<0.05) as determined by MaAsLin2. Red dots indicate species with increased relative abundance in ‘Flare’ compared to healthy controls (HG), household partners (HHP), or remission, while blue dots represent species with decreased relative abundance compared to other conditions. Dot size is proportional to the significance of the association, represented by the negative logarithm of the q-value. All detected associations are adjusted for households as random effects and disease status as fixed effects. (7B-7D) Relative abundance of top three species over-abundant in patients in a flare: Ruminococcus gnavus (7B), Escherichia coli (7C), and Klebsiella pneumoniae (7D).

FIGS. 8A-8C. (8A) Relative abundance of gut fungi at the phyla level in patients with clinically documented Auto-brewery Syndrome in remission or during a flare or their household partners (HHP). Each column represents one patient or household partner. (8B) Principal component analysis (PCoA) of the relative abundance of gut fungal species in patients with ABS in remission or flare or HHPs. P-value calculated by PERMANOVA. (8C) Representative figures of stool from a patient with ABS and their HHP cultured in YPD media and grown on YPD plates for 48 hours, next to a negative and positive control (Candida albicans).

FIG. 9 shows mixed acid and heterolactic fermentation pathways. Relative abundance of the Kyoto Encyclopedia of Genes and Genomes (KEGG) Orthology (KO) genes contributing to the mixed acid fermentation pathway and heterolactic fermentation pathway in patients with clinically documented Auto-brewery Syndrome in remission or during a flare or their household partners (HHP).

FIG. 10 shows relative abundance of the KEGG Orthology (KO) genes contributing to the ethanolamine utilization pathway in patients with clinically documented Auto-brewery Syndrome in remission or during a flare or their household partners (HHP).

FIG. 11 shows relative abundance of the Gene Ontology (GO)-annotated genes contributing to the ethanolamine utilization pathway, the heterolactic fermentation pathway, and the mixed acid fermentation pathway for ethanol production in healthy controls, patients with clinically documented Auto-brewery Syndrome in remission or during a flare or their household partners (HHP).

FIGS. 12A-12B. (12A) Stacked pathway enrichment contribution by mixed acid fermentation pathway, heterolactic fermentation pathway, acetylene degradation pathway, or ethanolamine utilization pathway in patients with clinically documented Auto-brewery Syndrome in remission or during a flare or their household partners (HHP). (12B) Relative attribution of bacterial species to the mixed acid fermentation pathway, heterolactic fermentation pathway, acetylene degradation pathway, or ethanolamine utilization pathway in patients with clinically documented Auto-brewery Syndrome in remission or during a flare or their household partners (HHP).

FIGS. 13A-13G. (13A) Ethanol produced by wild-type (WT) and ΔadhE E. coli in LB media supplemented with 200 mM glucose after 18 hours. (13B-13D) Portal vein ethanol levels (13B), serum ALT (13C), hepatic triglycerides (TG) (13D), representative images of Oil Red O and H & E staining of liver sections, scale bar=100 mM (13E), and hepatic Ccl2 (13F) and IIIb (13G) mRNA expression in gnotobiotic mice mono-colonized with wild-type or ΔadhE E. coli. Two independent experiments.

FIGS. 14A-14C. (14A) Timeline of biospecimen collection from the patient who received FMT, in relation to first FMT. (14B) Top 10 contributing species for axis 1 and 2 of the principal component analysis shown in FIG. 4 and % contribution. (14C) Longitudinal analysis of percent gut microbiota shared with initial pre-FMT sample, donor sample, and percent donor engraftment, over the course of two FMTs.

### DETAILED DESCRIPTION

Described herein are methods of diagnosis and/or treatment of Auto-brewery Syndrome (ABS), in which ABS patients can be statistically differentiated from cohabitating household controls, using in vitro bioreactor cultures and metagenomic taxonomic and functional analysis of fecal samples. Bioreactor and metagenomic findings are also confirmed using stool samples from one patient with Auto-brewery Syndrome who was successfully treated with fecal microbiota transplantation.

The present disclosure provides a comprehensive characterization of a cohort of patients with rigorously documented Auto-brewery Syndrome and their household partners, including untargeted gut microbiome analysis. One prominent barrier to the clinical diagnosis of Auto-brewery Syndrome is current standard of obtaining serial ethanol measurements over hours to days in a supervised clinical setting during a flare. This barrier to diagnosis of gut alcohol production conditions like ABS stands in the way of correctly identifying persons at risk of having a gut alcohol production conditions and effective treatment of the condition.

As described herein, bioreactor ethanol production by cultured fecal microbiota can accurately distinguish Auto-brewery Syndrome patients from asymptomatic household partners. The present disclosure also demonstrates that the predominant pathobionts responsible for Auto-brewery Syndrome in most patients are bacteria in the Proteobacteria phylum rather than fungi, as previously reported [8]. Additionally, known fermentation pathways are overabundant in the microbiomes of the majority of patients with Auto-brewery Syndrome, providing a molecularly consistent explanation for pathologic ethanol production by these patients' gut microbiota during symptomatic flares. This physiological explanation is further supported by the number of pathways involved in menaquinol biosynthesis that were over-represented in patients compared with their household partners, as menaquinone is important for the electron transport chain in bacteria, particularly in anaerobic conditions when fermentation occurs [29].

There is currently no consensus or standard therapy for Auto-brewery Syndrome, and unfortunately, patients with this debilitating condition can often suffer from delays in diagnosis, significant impairment in quality of life, and familial, social, and legal difficulties. Treatment options are also restricted by a lack of clear understanding of the underlying molecular pathologies. The present disclosure demonstrates successful treatment of a patient with Auto-brewery Syndrome using fecal microbiota transplantation and identified that the treatment is associated with a significant reduction in bacteria of the Proteobacteria phylum and fermentation pathway gene enrichment.

It is noted that as sed in the specification and the appended claims, the singular forms “a”, “an” and “the” refer to one or more (i.e., at least one) of the grammatical object of the article unless the context clearly dictates otherwise. By way of example, “a bacteria” encompasses one or more bacteria.

### Methods of Analyzing a Fecal Sample

Provided herein are methods of analyzing a fecal sample, wherein the fecal sample is both analyzed for the amount of ethanol the fecal sample produces and genetically analyzed with respect to either (a) the abundance of particular bacteria, or (b) the expression of genes associated with enzymes of the heterolactic fermentation pathway, the mixed acid fermentation pathway, the ethanolamine utilization pathway, acetylene degradation pathway, and/or menaquinol and demethylmenaquinol biosynthesis pathways. Such methods can be used for identification of a subject having or suspected of having a condition associated with gut alcohol production.

In some embodiments, the fecal sample is cultured under anaerobic conditions and the fecal sample culture is analyzed for ethanol production. Ethanol production of the fecal sample can be measured by any method known in the art, including but not limited to, chromatographic methods (e.g., gas chromatography, high performance liquid chromatography (HPLC)), spectrophotometric methods (e.g., dichromate oxidation, UV-vis spectrophotometry, near-infrared (NIR) spectrophotometry, or probe-based spectrophotometry), or enzymatic assays (e.g., ADH assay, or fluorometric assay). The ethanol production of the fecal sample can be required to be greater than a threshold amount before the fecal sample is subject to genetic analysis. The threshold can be 7.0-10.0 mg/dL (e.g., 7.5-8.5 mg/dL, 8.0-9.0 mg/dL, 8.5-9.5 mg/dL, or 9.0-10.0 mg/dL (e.g., 7.0 mg/dL, 7.5 mg/dL, 8.0 mg/dL, 8.5 mg/dL, 9.0 mg/dL, 9.5 mg/dL, or 10.0 mg/dL)). For example, the threshold can be 8.21 mg/dL. If fecal sample ethanol production reaches a certain threshold level, genetic analysis of the fecal sample can also be performed. For example, if ethanol production of the fecal sample is 8.21 mg/dL or higher (e.g., if the amount of ethanol produced by the fecal sample is higher than 8.21 mg/dL in bioreactor culture), the fecal sample is subject to genetic analysis.

Genetic analysis of the fecal sample can include shotgun metagenomic sequencing. The shotgun metagenomic sequencing can be in conjunction with (e.g., can be followed by) one or more methods for analysis (e.g., methods for statistical analysis and/or one or more bioinformatic tools). For example, the shotgun metagenomic sequencing can be in conjunction with (e.g., can be followed by) one or more statistical analysis methods like linear discriminant effect-size analysis (Lefse). Additionally, or alternatively, the shotgun metagenomic sequencing can be in conjunction with (e.g., can be followed by) use of one or more bioinformatic tools, e.g., to identify taxonomic composition of the gut microbiome samples (e.g., by using METAPHLAN) and/or to identify functional genomic composition of the gut microbiome samples (e.g., by using HUMANN).

In some embodiments, the genetic analysis does not include 16SrRNA sequencing. These types of genetic analyses can be used to identify the types of microbes in the fecal sample, the relative abundance of the microbes in the fecal sample as compared to each other, the relative abundance of the microbes as compared to a reference fecal sample, or the relative expression of genes implicated in ethanol production. For example, the genetic sequencing can be used to identify the abundance of Ruminococcus gnavus, Escherichia coli, or Klebsiella pneumoniae bacteria in the fecal sample. Relative abundance of these microbes (e.g., Ruminococcus gnavus, Escherichia coli, or Klebsiella pneumonia) in the fecal sample can be determined as compared to each other, and/or as compared to that in a reference fecal sample. As another example, genetic sequencing can be used to identify the amount or the relative expression of genes implicated in anaerobic bacterial ethanol production, such as, but not limited to fumarate reductase, pyruvate formate lyase, phosphotransacetylase, acetaldehyde dehydrogenase, alcohol dehydrogenase (ADH) (e.g., E. coli ADH), ethanolamine transporter, and/or ethanolamine ammonia-lyase. Relative expression of genes implicated in anaerobic bacterial ethanol production (e.g., fumarate reductase, pyruvate formate lyase, phosphotransacetylase, acetaldehyde dehydrogenase, alcohol dehydrogenase (ADH) (e.g., E. coli ADH), ethanolamine transporter, and/or ethanolamine ammonia-lyase) in the fecal sample can be determined as compared to each other, and/or as compared to that in a reference fecal sample. Any combination of the methods to measure microbial and/or anaerobic bacterial ethanol production and the genetic analysis methods described herein can be used to analyze a fecal sample. For use in the present methods, a reference sample (e.g., a reference fecal sample) can comprise the average of one or more fecal samples from a subject (or a population of subjects) not suspected of having a condition associated with gut alcohol production.

Methods of Treating a Condition Associated with Gut Alcohol Production

Provided herein are methods of treating a condition associated with gut alcohol production in a subject. The method can include one or more of the following steps: (a) analyzing a fecal sample obtained from a subject, wherein analyzing the fecal sample comprises culturing or having cultured the fecal sample and performing or having performed metagenomic sequencing of the fecal sample; (b) determining that the subject has a condition associated with gut alcohol production; (c) and if the subject has a condition associated with gut alcohol production, then administering to the subject an effective amount of a treatment. The treatment can comprise a probiotic (e.g., a probiotic with resistant starch), fecal microbiota transplantation (FMT), one or more antimicrobial agents, one or more microbial enzyme inhibitors, or a combination thereof.

The present methods can be used for treating a subject who has (e.g., is experiencing) one or more symptoms of a condition associated with gut alcohol production. The present methods can also be used for treating a subject who has experienced symptoms of a condition associated with gut alcohol production. For example, the present methods can be used for treating a subject who has experienced one or more (e.g., two or more, three or more, four or more, or five or more) episodes of symptoms of a condition associated with gut alcohol production within the last year. The present methods can also be used for treating a subject who is suspected of having a condition associated with gut alcohol production and/or has risk factors for a condition associated with gut alcohol production. In some embodiments, a subject is suspected of having a condition associated with gut alcohol production and a health history is performed. A health history can include detailed interview and/or questionnaires to determine the subject's past and current risk factors for a condition associated with gut alcohol production. Risk factors for a condition associated with gut alcohol production can include, but are not limited to, having three or more alcoholic drinks per week, having a prior history of antifungal use, and/or previous symptoms of a condition associated with gut alcohol production.

A condition associated with gut alcohol production can include one or more of Auto-brewery Syndrome (ABS), steatotic liver diseases, and alcohol use disorder. Steatotic liver diseases can include one or more of metabolic dysfunction-associated steatotic liver disease (MASLD) (formerly known as non-alcoholic fatty liver disease (NAFLD)), metabolic dysfunction and alcohol-associated liver disease (MetALD), and alcohol-associated liver disease (ALD). Thus, in some embodiments, the present methods can be used for treating ABS in a subject.

For treatment by the present methods, a fecal sample can be obtained from a subject. For example, a fecal sample can be obtained from a subject who has (e.g., who is experiencing) one or more symptoms of a condition associated with gut alcohol production. A fecal sample can also be obtained from a subject who has experienced symptoms of a condition associated with gut alcohol production. For example, a fecal sample can be obtained from a subject who has experienced one or more (e.g., three or more) episodes of symptoms of a condition associated with gut alcohol production within the last year. In some embodiments, a fecal sample is obtained from a subject, wherein the subject may or may not have risk factors for a condition associated with gut alcohol production.

A fecal sample obtained from a subject can be analyzed by one or more methods of the present disclosure. Analyzing a fecal sample can include culturing the fecal sample and/or sequencing the fecal sample as described herein.

The examples demonstrate a notable enrichment of genes involved in various menaquinol and demethylmenaquinol biosynthesis pathways in patient flare microbiome samples. Menaquinone and demethylmenaquinone are essential electron carriers for anaerobic ATP-generating redox reactions, and their concentrations are significantly higher in enterobacteria grown in anaerobic conditions compared with aerobic conditions [30]. This increased abundance, coupled with the elevated availability of electron acceptors, may contribute to the pathologic production of ethanol [31] in subject with a gut alcohol production condition.

The subject can be considered to have a condition associated with gut alcohol production if the fecal sample obtained from the subject produces at least 8.21 mg/dL ethanol. The subject can be considered to have a condition associated with gut alcohol production if the subject has a fecal sample ethanol level at least 1.3 times, 1.4 times, 1.5 times 1.6 times, 1.7 times, 1.8 times, 1.9 times, or 2.0 times that of a reference fecal sample. For use in the present methods, a reference sample can comprise the average of one or more fecal samples from a subject (or a population of subjects) not suspected of having a condition associated with gut alcohol production. Ethanol production of the fecal sample can be measured by any method known in the art, including but not limited to, chromatographic methods (e.g., gas chromatography, high performance liquid chromatography (HPLC)), spectrophotometric methods (e.g., dichromate oxidation, UV-vis spectrophotometry, near-infrared (NIR) spectrophotometry, or probe-based spectrophotometry), or enzymatic assays (e.g., ADH assay, or fluorometric assay).

In addition to determining the ethanol production of a fecal sample from the subject, the fecal sample can be subject to sequencing analysis, wherein the sequencing analysis can be used to determine (a) the types of microbes present in the fecal sample, (b) the relative abundance of particular microbes in the fecal sample, (c) the expression or relative representation of biochemical pathways expressed in the fecal sample, and/or (d) the expression or relative representation of genes expressed in the fecal sample.

Culturing the fecal sample and obtaining a measurement of ethanol production from the fecal sample provides a functional readout (e.g., physiological evidence) of the fecal sample genetic sequencing analysis. The genetic sequencing analysis (e.g., metagenomic sequencing provides a broader and non-biased analysis of the microbial members of the fecal sample and how the microbial members of the fecal sample may be interacting with each other to produce the functional result (e.g., ethanol production). Additionally, the metagenomic sequencing provides information as to an effective therapeutic approach.

### Therapeutic Treatment

A subject determined to have a condition associated with gut alcohol production can be administered a therapeutic treatment. The therapeutic treatment can comprise a probiotic (e.g., a probiotic with optional resistant starch), fecal microbiota transplantation (FMT), one or more antimicrobial agents, one or more microbial enzyme inhibitors, or a combination thereof.

A subject determined to have a condition associated with gut alcohol production can be administered a probiotic with optional prebiotic. Probiotic microorganisms are a type of live microbial food ingredient which is beneficial to health and may selectively stimulate the growth of native bacteria in the intestinal tract. Probiotic microorganisms have been reported to exert effects such as inhibiting the growth of pathogens in the gastrointestinal tract, alleviating lactose intolerance, improving immunoregulatory function, providing anti-cancer properties, and lowering blood pressure. Examples of common probiotic microorganisms include bacterial strains from Lactobacillus, Bifidobacterium, Bacillus, Lactococcus, Enterococcus, Saccharomyces, and Streptococcus. Probiotics may be administered with other agents used to aid in the effectiveness of the probiotic. For example, a probiotic may be administered with a prebiotic. Prebiotics are a nondigestible fibers that acts as a food source for probiotics or otherwise promotes the growth of beneficial microbes. One example of a prebiotic is a resistant starch (e.g., RS1, RS2, RS3, or RS4). Resistant starch is a type of fiber that ferments in the gut, providing a source of food for probiotics. For example, a subject determined to have a condition associated with gut alcohol production can be administered a probiotic with optional resistant starch.

A subject determined to have a condition associated with gut alcohol production can be administered one or more probiotics and/or prebiotics according to a predetermined schedule. For example, the predetermined schedule can be at least once daily (e.g., twice daily, three, four, or five times daily) for a determined time period. The determined time period can be at least one week, at least two, three, or four weeks. The determined time period can be at least one, two, three, four, five, or six months.

A subject determined to have a condition associated with gut alcohol production can be administered a fecal microbiota transplant (FMT). Fecal microbiota transplantation (FMT), sometimes known as fecal transplant, is a procedure that collect feces from a healthy donor and introduces them into another subject's gastrointestinal tract. FMT can be performed by colonoscopy, wherein a medical professional guides the colonoscope through the colon and as the colonoscope is withdrawn a fecal solution from the healthy donor is deposited into the subject's gastrointestinal tract. Other methods of fecal transplantation can include administration of fecal microbes through a nasal cannula that reaches the duodenum thereby depositing the microbes in the duodenum, orally administered fecal microbe-containing capsules which are designed to bypass the stomach and dissolve in the colon, or by enema.

A fecal sample prepared for an FMT may be analyzed prior to transplantation. The analysis may be to screen the sample to ensure it is free of pathogens and other harmful substances that could be transmitted to the recipient. A fecal sample may also be screened for presence of desirable microbes as a basis for the selection of a particular fecal sample prior to administration as FMT. For example, the fecal sample may be screened for acetate-producing bacteria (e.g., bifidobacterim, lactobacillus, clostridium, akkermansia, bacteroides, prevotella, or actinobacteria) prior to FMT preparation, wherein fecal samples containing acetate-producing bacteria are selected for FMT. The examples show a significant enrichment of genes involved in the tricarboxylic (TCA) cycle from acetate-producing bacteria in remission samples compared with flare samples from the same subject. This suggests enhanced ethanol metabolism by acetate-producing bacteria may contribute to the spontaneous remission of auto-intoxication symptoms observed in some patients, potentially triggered by shifts in the individual's microbiome.

FMT can be administered to the subject determined to have a condition associated with gut alcohol production according to a predetermined schedule (e.g., one or more times). The FMT may be administered according to a schedule (e.g., multiple times in one week, once a week, once a month) until the transplant has been determined to be effective. A fecal transplant can be determined to be effective through monitoring of ethanol production of the subject's fecal matter, self-reported or observed symptoms of auto-intoxication, fecal genomic sequencing, or a combination thereof. For example, the fecal transplant may be determined to be effective if the subject's fecal sample produces less than 8.21 mg/dL ethanol in a bioreactor culture, or if genomic sequencing of the subject's gut microbiota post-FMT closely resembles that of the FMT donor's gut microbiota. Administration of FMT can be combined with administration of probiotics for the treatment of a gut alcohol production condition in a subject. For example, a subject can be administered FMT one or more times in conjunction with regular administration of a multi-strain probiotic in conjunction with resistant starch.

One or more antimicrobial agents can be administered alone, before, or during FMT in a subject determined to have a condition associated with gut alcohol production. For example, upon determination that the subject has a condition associated with gut alcohol production, the subject can be administered antimicrobial agent(s) to treat the condition associated with gut alcohol production. The antimicrobial agents can be antibacterial agents and/or antifungal agents. For example, the antibacterial agent(s) can be selected to target particular bacterial strains determined to be present in the subject's fecal sample. In some embodiments, the subject can be administered antimicrobial agent(s) before an anticipated FMT in order to prepare the subject's gastrointestinal tract to receive the FMT. Treatment of a subject determined to have a condition associated with gut alcohol production can include administration of one or more of antimicrobial agent(s), FMT, prebiotics, and probiotics. For example, a subject can be administered antimicrobial agent(s) before an anticipated FMT, undergo one or more administrations of FMT according to a schedule, and then be administered pre- and/or probiotics on an ongoing basis.

The antimicrobial agent(s) can include one or more antibacterial agents. For example, the antibacterial agent(s) can be selected to target ethanol producing bacteria in the fecal sample. In some embodiments, the antibacterial agent(s) target E. coli and comprise one or more of fluoroquinolones (e.g., ciprofloxacin or levofloxacin), beta-lactams (e.g., amoxicillin or ceftriaxone), trimethoprim/sulfamethoxazole, nitrofurantonin, rifaximin, macrolides, ampicillin, cefdinir, and fosfomycin. In some embodiments, the antibacterial agent(s) target Ruminococcus gnavus and comprise one or more of penicillin, piperacillin-tazobactam, ampicillin, cefotaxime, metronidazole, imipenem, meropenem, clindamycin, tetracycline, and vancomycin. In some embodiments, the antibacterial agent(s) target Klebsiella pneumoniae and comprise one or more of cephalosporins (e.g., ceftazidime, ceftriaxone, or cefepime), fluoroquinolones (e.g., ciprofloxacin), aminoglycosides, carbapenems (e.g., meropenem, imipenem, or ertapenem), cefiderocol, colistin, polymyxin-B, tigecycline, plazomicin, doxycycline, and ceftazidime-avibactam.

Treatment of a subject determined to have a condition associated with gut alcohol production can include one or more antifungal agents. For example, one or more antifungal agents can be used to treat a subject if metagenomic sequencing of fecal sample of the subject shows evidence of fungal contribution to ethanol production (e.g., high relative abundance of Saccharomyces cerevisiae and/or Candida species in the fecal sample of the subject). Thus, in some embodiments, a subject determined to have a condition associated with gut alcohol production is treated with a combination of antibacterial agents and antifungal agents. Antifungal agents for use in the present methods can include one or more of azoles (e.g., fluconazole, itraconazole, and Posaconazole), allylamines (e.g., terbinafine), polyenes (e.g., amphotericin B and nystatin), echinocandins, flucytosine, and griseofulvin.

A subject having a condition associated with gut alcohol production can be treated according to the methods disclosed herein by targeting ethanol metabolism of anaerobic microbes. Microbial enzyme inhibitors can be administered to a subject determined to have a condition associated with gut alcohol production, wherein the microbial enzyme inhibitors are directed to suppress a biochemical pathway associated with anaerobic bacterial ethanol production. For example, the microbial enzyme inhibitor can inhibit an enzyme in the heterolactic fermentation pathway, the mixed acid fermentation pathway, the ethanolamine utilization pathway, acetylene degradation pathway, and/or menaquinol and demethylmenaquinol biosynthesis pathways. In some non-limiting embodiments, the microbial enzyme inhibitor is thiabendazole, cambendazole, fenbendazole, oxfendazole, methacrylate, acryalate, metadoxine, disulfiram, fomepizole, or combinations thereof.

The microbial enzyme inhibitors can be administered according to a predetermined schedule. For example, the predetermined schedule can be at least once daily (e.g., twice daily) for a determined time period. The determined time period can be at least one week, at least two, three, or four weeks. The determined time period can be at least one, two, three, four, five, or six months. The administration of microbial enzyme inhibitors can be alone or in conjunction with another therapy disclosed herein (e.g., prebiotics and probiotics, FMT, and antimicrobial agents).

### EXAMPLES

The invention is further described in the following examples, which do not limit the scope of the invention described in the claims.

### Example 1. Materials and Methods

### Study Design and Study Population

A prospective, observational, study of patients with Auto-brewery Syndrome was performed, including serial stool and blood sampling. Patients were recruited via the Auto-Brewery Syndrome Information and Research, Inc. non-profit 501(c)3 organization and their support groups. Inclusion criteria for patients with Auto-brewery Syndrome include age over 18 years and measured increased blood alcohol concentration, either spontaneously or after exposure to oral glucose challenge, while the patient is supervised in a clinical setting without alcohol consumption. Inclusion criteria for household partners (HHP) were that they lived with a confirmed patient with ABS in the same household. Eligible persons were at least 18 years of age. Patients with Auto-brewery Syndrome included in our study all had documentation of serial rise in blood alcohol concentration as measured by serum ethanol levels (86%) or breathalyzer (14%) while in a supervised clinical setting (or in their home in the presence of a medical professional for one participant) in the documented absence of alcohol consumption. Patients who had obtained a diagnosis of Auto-brewery Syndrome by other diagnostic methods such as culture of duodenal aspirate or stool culture but did not have documented rise of blood alcohol concentration in a supervised clinical setting in the absence of alcohol consumption were excluded from the study. Patients' household partners (HHPs), who did not exhibit any symptoms of Auto-brewery Syndrome, were also enrolled and dyads were linked by a household ID. Within the recruited patient pool, four pairs of individuals were studied at baseline and then re-enrolled under discrete, different household IDs after receiving significant microbiome-altering agents. One of these four patients (A 20) underwent fecal transplant as treatment as part of this study and was studied over time for 4 years. The other three pairs of individuals received broad-spectrum antibacterial and antifungal agents and experienced relapse 3, 10, and 26 months after antimicrobial treatment, respectively.

Baseline demographic data and laboratory test results were collected and a comprehensive clinical history was obtained from each participant. Fasting blood samples were collected; serum and plasma samples were frozen and stored at −80° C. until use. At least 10 grams of stool were collected from each patient during remissions and flares of clinical intoxication. Clinical intoxication describes a condition based off clinical symptoms that include changes in cognition, perception, coordination, and behavior. The blood alcohol concentration necessary to produce symptoms of clinical intoxication differs by individual, and in our case, it is defined by the presence of both symptoms of intoxication and a positive alcohol breath test. Samples were collected from household partners within 24 hours of patient samples. Concurrent blood alcohol concentrations were measured by an FDA-approved portable breathalyzer (BAC track, San Francisco, CA), and results were recorded by the patient. Stool samples were immediately frozen in the patient's freezer and shipped within 24-48 hours on dry ice to UC San Diego and stored at −80° C. until use. The Auto-brewery Syndrome Registry was approved by the UCSD Institutional Review Board, and written informed consent was obtained from each participant.

### Fecal Microbiota Transplantation (FMT)

Subject A 20 and his household partner connected the research groups requesting consideration of fecal transplant, as his Auto-brewery Syndrome had begun after multiple courses of antibacterial therapy for urinary tract infections prompted by prostate enlargement which was ultimately surgically corrected. Empiric antifungal therapy, virtual total elimination of dietary carbohydrates and alternative/complementary medical treatments had failed to durably suppress this subject's ongoing flares of documented intoxication with serious medical and social consequences. IRB and FDA approval (Single Patient Expanded Access IND 28390/PI Hohmann) was obtained for single donor, capsule-based fecal transplant, performed after written informed consent was obtained. The subject received oral antibiotic pre-treatment (rifaximin, erythromycin and neomycin for 3 days) and a standard pre-colonoscopy purge with polyethylene glycol solution to empty the GI tract of food and stool, prior to administering 5 doses of 15 FMT capsules over a week's time. The subject did well for 3 months with marked improvement in health status (by both self and family reports), and absence of intoxication flares, but relapsed thereafter. Repeat FMT was proposed to regulators, this time pre-treating with vancomycin, metronidazole and trimethoprim/sulfamethoxazole orally for 3 days, followed by a purge. On repeat treatment, the subject received 3 doses of 15 capsules after pre-treatment, followed by monthly maintenance dosing of 15 capsules (derived from the same donor) without pre-treatment for 6 months. FMT capsules were generated as previously described [9] from the same healthy unrelated donor, and the subject was NPO for 3 hours before and one hour after dosing. The patient was also directed to take one heaping tablespoon daily of potato starch (Bob's Red Mill, Milwaukie, OR) dissolved in water [10], and a commercially available combination probiotic, one capsule daily, containing Bifidobacteria intantis, Clostridium butyricum, Clostridium beijerinckii, Anaerobacterium halii and Akkermansia muciniphilia (Metabolic Daily; Pendulum, San Francisco CA) in an effort to promote persistence of the transplanted microbiome.

### Bioreactor Cultures

100 mg of stool collected from patients during flare and remission and from their household partners was cultured in bioreactor cultures which consisted of 10 mL of Luria Broth (LB) media supplemented with 2 mM glucose and 2 mM cysteine in anaerobic conditions using hungate tubes. This concentration of glucose was chosen to emulate physiologic glucose concentrations in the intestine [11]. Bioreactor cultures were untreated or treated with 0.05% dimethyl sulfoxide (DMSO, vehicle), 10 μg/mL amphotericin B, or 100 μg/mL chloramphenicol. To emulate supraphysiologic concentrations of glucose in the GI tract, patient microbiota was also cultured in LB media supplemented with 100 mM and 200 mM of glucose anaerobically using hungate tubes. Samples were collected at 0, 6, and 24 hours of culturing and ethanol concentration was assessed by high-performance liquid chromatography (HPLC) using a 1260 Infinity IILC system (Agilent, Santa Clara, CA, USA) equipped with an HPX-87H (Biorad, Hercules, CA, USA) or by using an enzyme-based kit (Abcam, in less than 20% of samples, low limit of detection 50 ng/dL).

### Bacterial Strain Isolation

To isolate high ethanol-producing bacterial strains, fecal samples from patients with Auto-brewery Syndrome were cultured in Luria Broth medium supplemented with 5% ethanol to select for bacteria capable of growth in high ethanol concentrations, under anaerobic and aerobic conditions at 37° C. for 24 hours, then plated onto agar plates. 100 single colonies were isolated at random, grown in 5 mL of LB media for another 24 hours, and DNA was isolated using the QIAamp® Fast DNA Stool Kit (Qiagen) according to the manufacturer's instructions. Briefly, bacteria were first pelleted by centrifugation, treated with a buffer and homogenized thoroughly, then heated at 95° C. to lyse cells. Proteins were digested with the addition of Proteinase K and Buffer AL, and incubated at 70° C. for 10 minutes. Ethanol was then added to the lysate to precipitate the DNA, and the resulting lysate was applied to a spin column and centrifuged to bind the DNA. The column was washed with Buffer AW1 and AW2, and then centrifuged to remove residual wash buffer before elution with sterile water. After quantification of DNA concentration by spectrophotometry analysis, PCR amplification was performed using universal 16 S PCR primers and Platinum PCR supermix (Invitrogen). PCR amplicons were purified using the QIAquick PCR Purification Kit (Qiagen) according to the manufacturer's instructions. Briefly, 5 volumes of Buffer PB were added to 1 volume of the PCR reaction mixture, and the mixture was adjusted to a yellow color by adding 10 μl of 3 M sodium acetate (pH 5.0) if necessary. The sample was then applied to a QIAquick column and centrifuged at 30-60 s or subjected to vacuum until the flow-through was complete. The column was washed with 750 μl of Buffer PE and centrifuged again to remove excess wash buffer. After a final centrifugation step to remove residual wash buffer, the purified DNA was eluted from the column using 50 μl of Buffer EB (10 mM Tris·Cl, pH 8.5) or water (pH 7.0-8.5) and centrifuged for 1 min. For gel analysis, the purified DNA was mixed with 1 volume of Loading Dye and 5 volumes of purified DNA, and the solution was loaded onto the gel after mixing. Purified PCR amplicons were submitted for Sanger sequencing and bacterial identities were determined by comparing 165 sequences to two public databases, NCBI Microbial Blast and the Ribosomal Database Project (RDP).

### Fungal Colony Isolation

To assess the presence of fungal colonies in stool microbiota, 30-50 mg of stool from patients and HHPs were resuspended in Y east Extract Peptone Dextrose (YPD) media at a 1% (w/v) concentration in culture tubes, vortexed to ensure thorough homogenization, incubated at 30° C. for 4 hours, and plated onto YPD agar plates supplemented with 100 μg/mL chloramphenicol and 100 μg/mL gentamicin to inhibit bacterial growth. The plates were incubated for 48 hours at 30° C. and fungal colonies were assessed. Candida albicans was separately plated as a positive control.

### Metagenomic Sequencing and Analysis

50 mg of stool from patients with Auto-brewery Syndrome in remission and flare, their household partners, and healthy controls were submitted for metagenomic sequencing (minimum of 1 million reads per sample) by the UC San Diego Microbiome Core. The Microbiome Core performed nucleic acid extractions utilizing previously published protocols [12]. Briefly, samples were purified using the MagMAX Microbiome Ultra Nucleic Acid Isolation Kit (Thermo Fisher Scientific, USA) and automated on KingFisher Flex robots (Thermo Fisher Scientific, USA). Blank controls and mock communities (Zymo Research Corporation, USA), which included Listeria monocytogenes, Pseudomonas aeruginosa, Bacillus subtilis, Escherichia coli, Salmonella enterica, Lactobacillus fermentum, Enterococcus faecalis, Staphylococcus aureus, Saccharomyces cerevisiae, and Cryptococcus neoformans, were included per extraction plate, which were carried through all downstream processing steps. DNA was quantified using a PicoGreen fluorescence assay (Thermo Fisher Scientific, USA) and metagenomic libraries were prepared with KAPA HyperPlus kits (Roche Diagnostics, USA) in a miniaturized ⅕th reaction volume format, and automated on EpMotion automated liquid handlers (Eppendorf, Germany). Sequencing was performed on the Illumina NovaSeq X Plus sequencing platform with paired-end 150 bp cycles at the Institute for Genomic Medicine (IGM), UC San Diego. A total of 87 samples were sequenced, including 21 samples from household partners, 20 samples from patients with ABS in remission, 25 samples from patients during a flare, and an additional 21 samples from our FMT patient and household partner during and after FMTs. 1-5 million reads were generated per sample. Three patients with ABS provided more than one sample during the flare condition and these samples were averaged for each patient. Samples were not averaged for patients who provided only one sample during remission and one sample during flare. All household partners contributed one sample each and their samples were not averaged.

To account for the potential contribution of sex-differences between patients with Auto-brewery Syndrome and their household partners, published gut microbiome metagenomic sequencing data from age, sex, and BMI-matched healthy controls from the United States was downloaded and analyzed as an additional comparator for microbial taxonomic and functional analyses. Briefly, the metadata for healthy controls enrolled through the iMSMS Consortium [13] was downloaded through the NCBISRA (accession number PRJEB32762) and optimal pair matching was performed using the “Matchlt” package in R for the variables of age, sex, and BMI. The published metagenomic sequencing data from these 22 matched individuals were downloaded and analyzed as below.

Raw metagenomic reads were pre-processed with KneadData (KneadData version 0.12.0, Trimmomatic version 0.33, Bowtie version 2.5.3), for quality control, filtering, and removal of contaminating host (human) and viral reads [14]. Taxonomic and functional profiling was performed using the HUMAnN 3 (version 3.8) pipeline [14] with MetaPhlAn4 (version 4.0.6, database version mpa_vOct22_CHOCOPhIAnSGB_202212) [15] and Diamond (version 2.1.9, full UniRef90 database). MaAsLin2 was used to identify associations between host phenotype and gut microbial taxonomic and metagenomic features [16]. Patients and their household partners were grouped into households and the households' random effects were controlled for. To identify gut microbial species associated with disease state, MaAsLin2 was used with a minimum abundance threshold of 0.00001 and a minimum prevalence of 10%. To identify microbial metabolic pathways associated with disease state, MaAsLin2 was used with a minimum abundance threshold of 0.00001 and a minimum prevalence of 20%. P-values were corrected using the Benjamini-Hochberg method to control for the false discovery rate and q-values >0.05 were considered statistically significant. Sequence reads will be deposited to NCBI.

Internal Transcribed Spacer (ITS) rRNA Sequencing and Analysis

The UC San Diego Microbiome Core performed nucleic acid extractions on stool samples from patients with Auto-brewery Syndrome in remission and flare and their household partners using the same protocol as for metagenomic sequencing. The ITS2 region was amplified using primers ITS3-2024F, the product was purified, the libraries were prepared and sequenced using the Illumina NovaSeq 6000 platform, and ITS2 amplicon sequences were demultiplexed and adapters were removed (Novogene). The quality of the sequences was assessed via FastQC [17]. The FastQC reports were used to determine where reads should be trimmed, and the reads were trimmed and filtered with BBduk [18]. After trimming, the DivisiveAmplicon Denoising Algorithm 2 (DADA2 version 1.26) pipeline [19] was used via the RStudio environment to assign reads to amplicon sequence variants using the UNITE fungal ITS database (version 10, published on Apr. 4, 2024) as the reference. The compositional relative abundance across samples was calculated and fungal genera associations were compared across the patient groups using Maaslin2 [16]. The arcsine square root transformation was used by Maaslin2 to transform the fungal genera relative abundances before running a linear mixed effects model. A minimum abundance threshold of 0.00001 and a minimum prevalence of 20% was used. To analyze beta diversity of the fungal composition, Aitchison distances were calculated by transforming amplicon sequence variant counts with a center-log ratio transformation, then calculating the Euclidean distance. Aitchison distance was input for principal coordinates analysis (PCoA). Permutational multivariate analyses of variance (PERMANOVA) were performed to determine if there were significant differences in microbial composition across groups.

### Gnotobiotic Mice

C57B L/6 germ-free mice were bred at UCSD. Female mice were colonized with wild-type or isogenic E. coli lacking alcohol dehydrogenase E (ΔadhE E. coli) [20] (kindly provided by Dr. Hirotada Mori) between 4-6 weeks of age and fed chow diet and water. After one week, mice continued to receive chow diet and were switched to sterilized water containing 2% glucose. After 10 weeks, mice were sacrificed and tissues were extracted. Animals were maintained on a 12 h:12 h light-dark cycle in Sentry SPP systems (Allentown, NJ) under gnotobiotic conditions. All manipulations were performed during the light cycle. All animal studies were reviewed and approved by the Institutional Animal Care and Use Committee at UCSD.

### Statistical Analysis

All statistical analyses and data visualization were performed using R statistical software (R version 4.2.2; R Foundation for Statistical Computing) within the RStudio integrated development environment. Continuous patient characteristics are presented as median and interquartile range. The Fisher exact test and Kruskal-W allis test were used to compare groups for binary and continuous variables, respectively. All statistical tests were two-sided, and a p-value equal to or less than 0.05 was considered statistically significant. Optimal cut-points for binary classification tasks were determined using the R package “cutpointr” to maximize the sum of sensitivity and specificity [21].

### Patient Characteristics

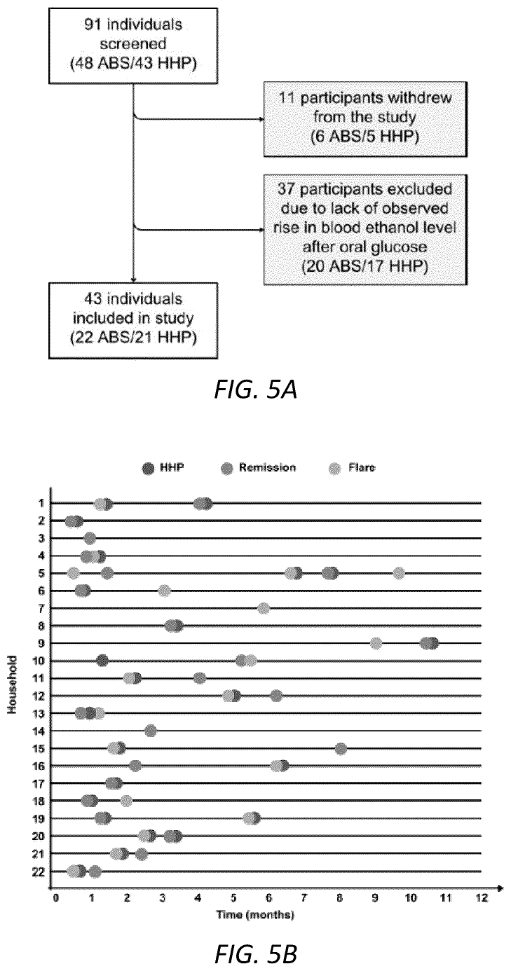

A total of 91 subjects (48 Patients who reported symptoms of Auto-brewery Syndrome and 43 household partners) were screened. 22 individuals with clinically documented Auto-brewery Syndrome diagnosed by spontaneous rise in blood alcohol level after oral glucose challenge and 21 unaffected household partners were enrolled in our study (FIG. 5A). One patient lived alone. During diagnostic testing, the median peak serum alcohol concentration after oral glucose load was 73 mg/dL (IQR 41.75-142 mg/dL) and occurred 240 minutes after oral glucose load (IQR 120-420 minutes). Patients with Auto-brewery Syndrome were more likely to be male and exhibited serum markers suggestive of liver injury, such as elevations in alanine transaminase (ALT), aspartate transaminase (AST), alkaline phosphatase (AP), and total bilirubin, compared with household partners. Patients with Auto-brewery Syndrome were also more likely to consume three or more alcoholic drinks per week prior to diagnosis, and to have a prior history of antifungal use, and less likely to consume foods rich in starch after diagnosis (Table 2). 16 households provided stool samples from the household partner, patient during remission, and during flare. “Flare” was defined as periods when patients had symptoms of intoxication or had a detectable blood alcohol level. “Remission” was defined as having no symptoms nor detectable blood ethanol levels for at least one week. Of the remaining 6 households, five patients did not develop a flare during the duration of their enrollment and one patient did not provide a sample during remission (FIG. 5B). The average blood alcohol concentration measured by breathalyzer during collection of stool samples during a flare was 136+/−82 mg/dL, significantly above the US legal driving limit of 80 mg/dl.

### Example 2. Microbial Ethanol Production in Bioreactor Cultures is a Diagnostic Marker of Auto-Brewery Syndrome

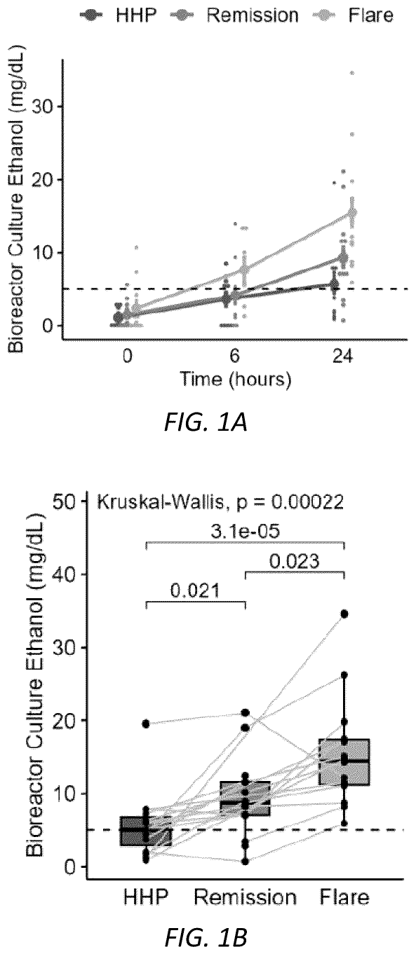

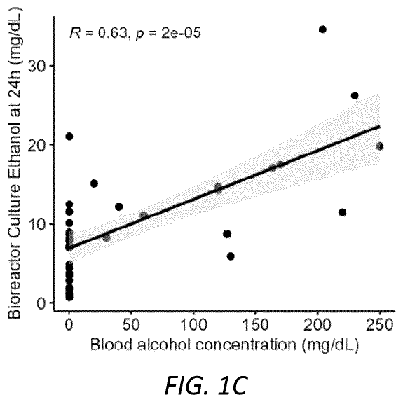

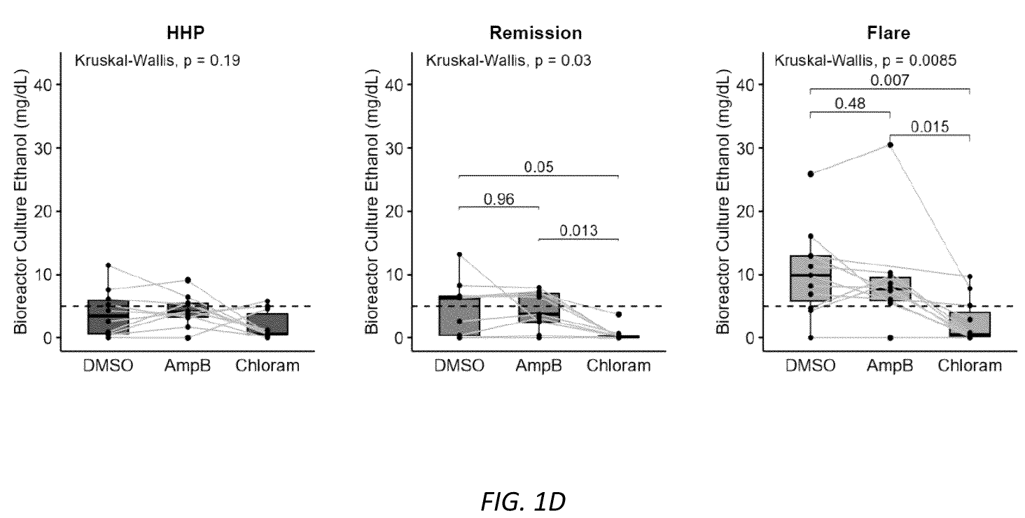

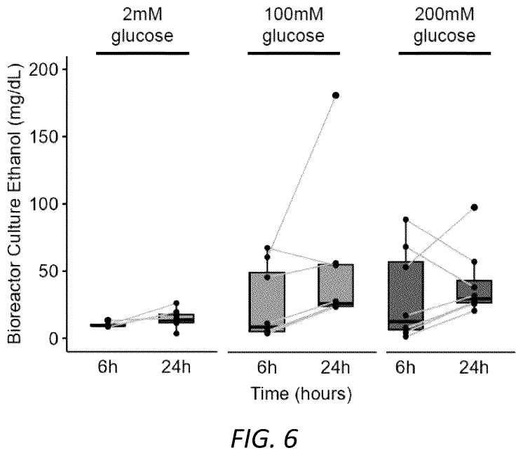

Stool samples from patients with clinically documented Auto-brewery Syndrome during a flare produced more ethanol in bioreactor cultures, with a median of 14.47 mg/dL (IQR 11.18, 17.37), compared with stool samples from patients with clinically documented Auto-brewery Syndrome in remission (median 8.76 mg/dL, IQR 7.03, 11.55) or from household partners (median 5.00 mg/dL, IQR 2.89, 6.79) at 24 hours of growth (FIGS. 1A, 1B). Using the calculated optimal cut-off of 8.21 mg/dL of ethanol production in bioreactor culture successfully discriminated stool samples obtained from patients with Auto-brewery Syndrome during a flare from their household partners with a sensitivity of 0.929, specificity of 0.933, and AUC of 0.919, and successfully discriminated stool samples obtained from patients with Auto-brewery Syndrome at any point from their household partners with a sensitivity of 0.759, specificity of 0.933, and AUC of 0.830. Blood alcohol concentration of flaring Auto-brewery Syndrome subjects at the time of stool collection positively correlated with ethanol production in bioreactor cultures at 24 hours (FIG. 1C). Ethanol fermentation in culture rose accordingly when patient microbiota was cultured in higher concentrations of glucose, reaching levels over 50 mg/dL (FIG. 6).

Treatment of bioreactor cultures with the broad-spectrum antibiotic chloramphenicol significantly decreased ethanol production in samples from patients with clinically documented Auto-brewery Syndrome, while treatment with the broad-spectrum antifungal amphotericin B did not have a significant effect (FIG. 1D) when compared with the DMSO vehicle control. However, ethanol production was partially reduced by amphotericin B in three Auto-brewery Syndrome patient samples during a flare (FIG. 1D). No significant difference in ethanol production was observed in bioreactor cultures of household partner samples treated with amphotericin B or chloramphenicol (FIG. 1D).

### Example 3. Patients with ABS Exhibit Gut Dysbiosis

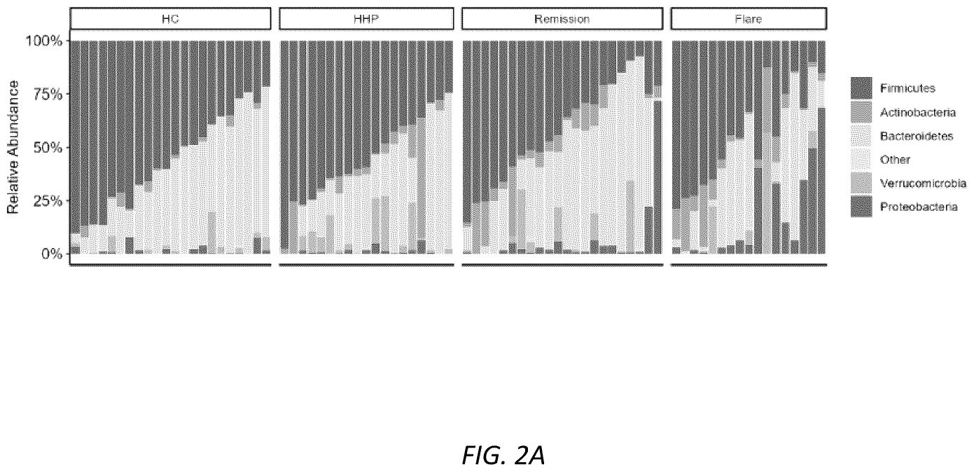

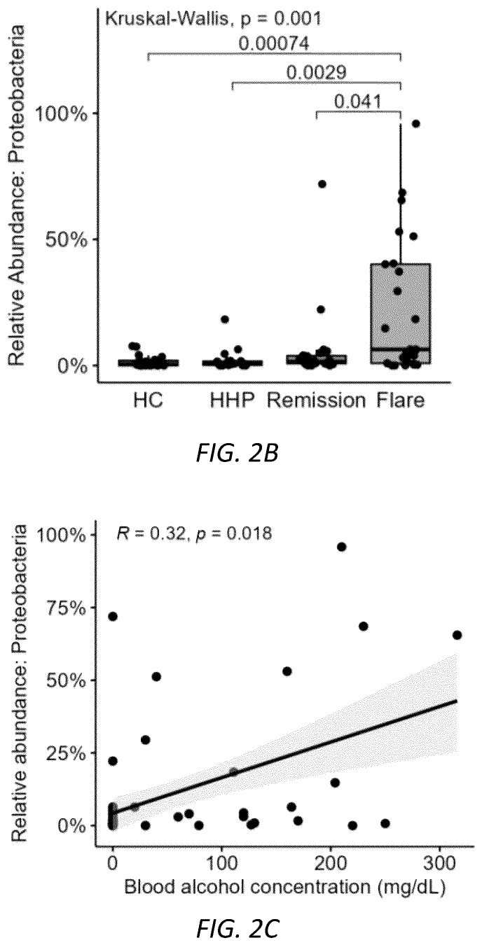

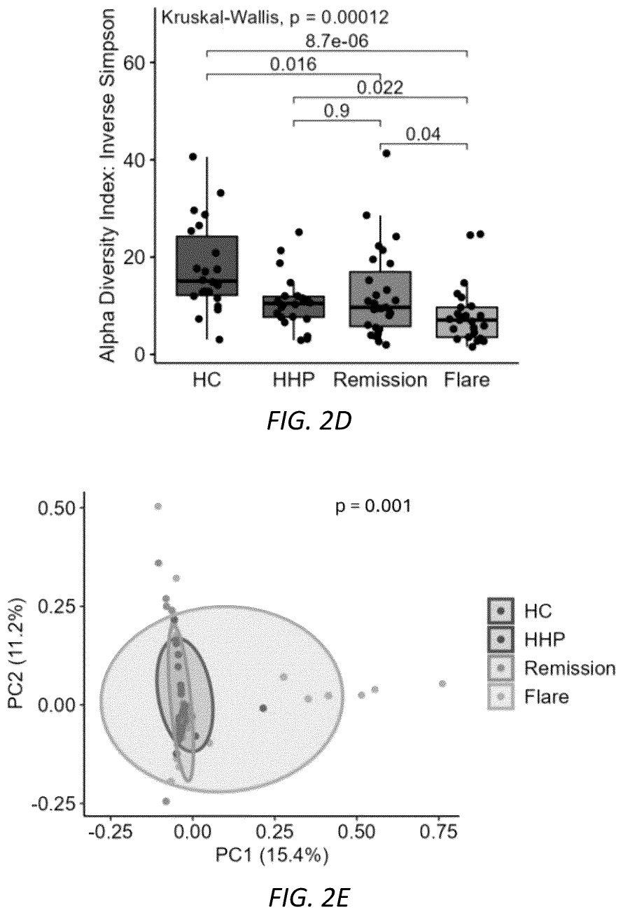

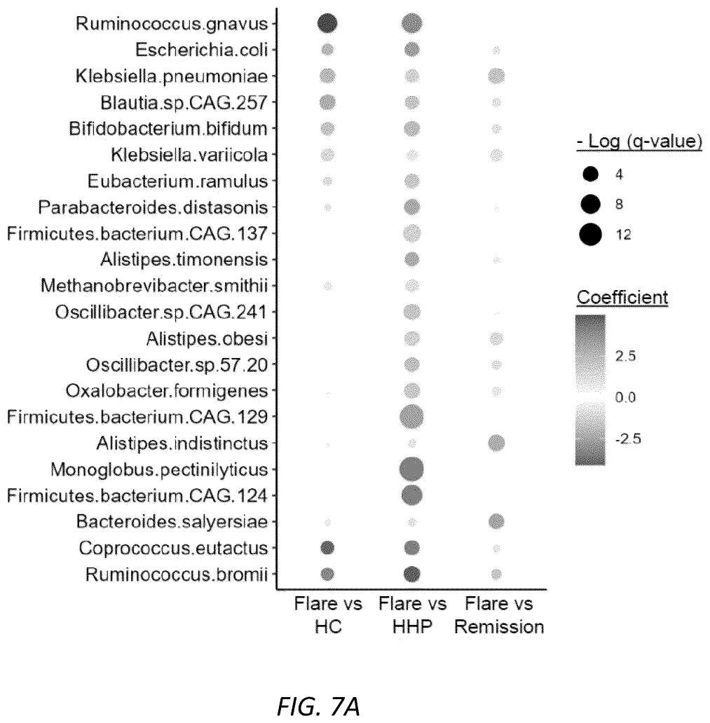

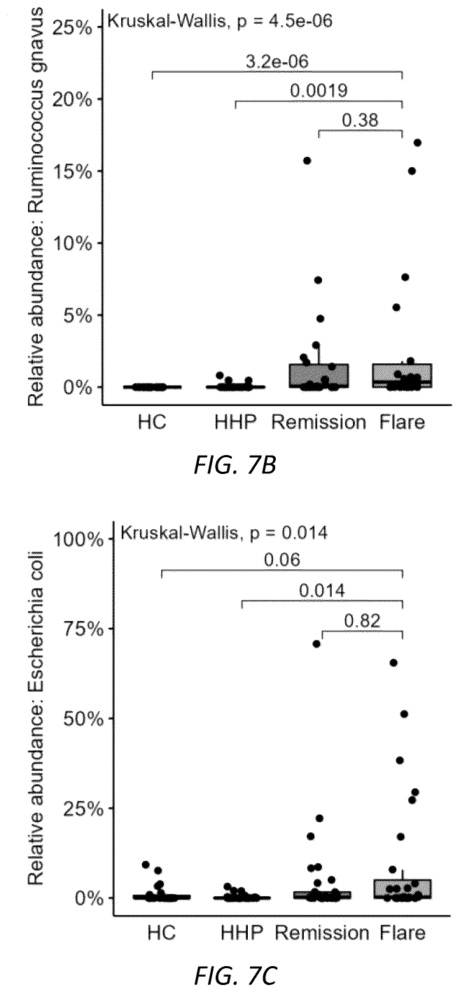

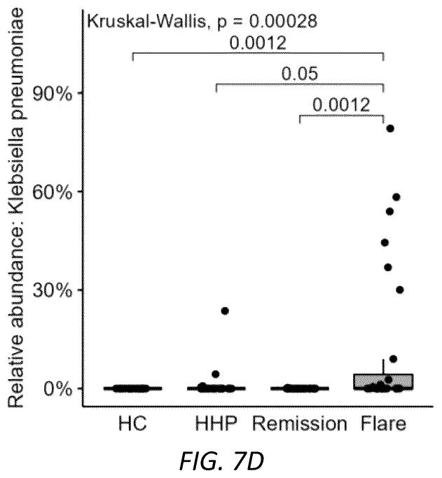

Analysis of shotgun metagenomic sequencing data demonstrated that the gut bacteria of patients with Auto-brewery Syndrome during a flare contained a significantly higher relative abundance of the Proteobacteria phylum as compared with samples from periods of remission and samples from household partners or healthy controls (FIG. 2A, B). Blood alcohol concentration of subjects at the time of flares with stool sample collection positively correlated with the relative abundance of the Proteobacteria phylum within the sample (FIG. 2C). Significant species differences (q-value <0.05) identified between the different conditions by MaAsLin2 while controlling for the random effects of each household are shown in FIG. 7A. The top three most differentially over-abundant bacterial species during a flare were Ruminococcus gnavus, Escherichia coli, and Klebsiella pneumoniae (FIGS. 7B-7D). Ruminococcus gnavus is thought to be a pathogenic bacteria associated with metabolic dysfunction-associated steatotic liver disease (MASLD) and liver cancer [22], while Escherichia coli and Klebsiella pneumoniae are members of the Proteobacteria phylum with known ethanol fermentation capacity. Also notably, several obligate anaerobic bacteria within the Clostridiales order, such as Ruminococcus bromii and Coprococcus eutactus, were significantly less abundant during a flare as compared with other disease states (FIG. 7A). These species within the Firmicutes phylum are able to metabolize sugars into short chain fatty acids, such as acetate and butyrate, which have been demonstrated to promote gut barrier integrity [23].

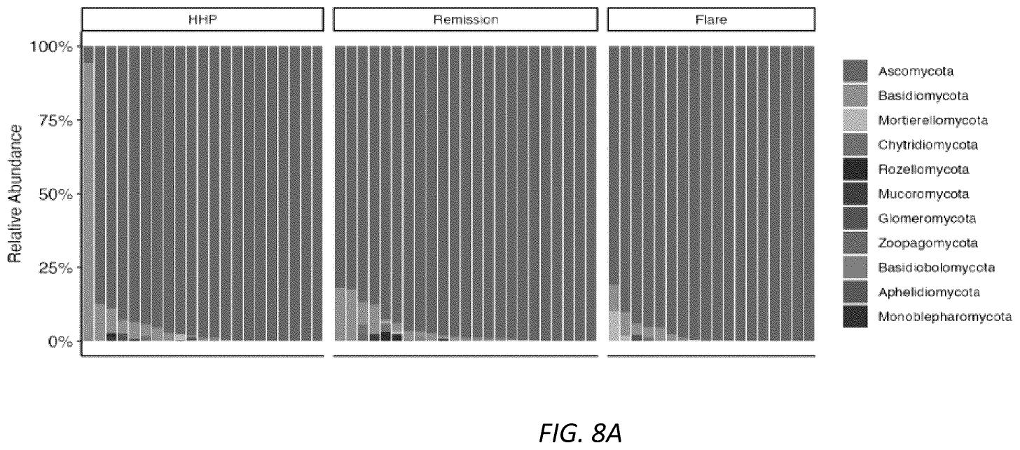

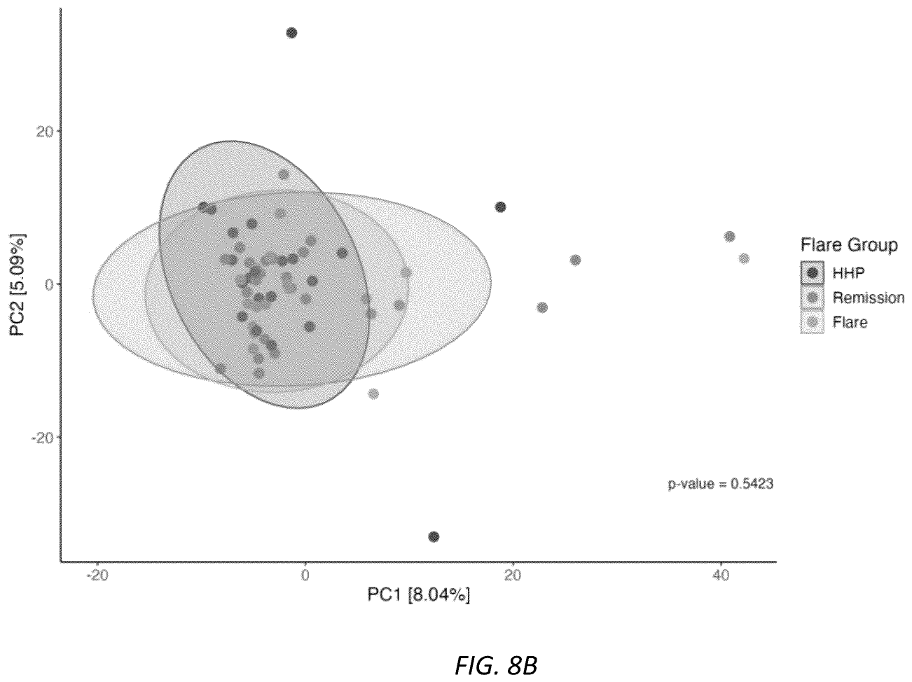

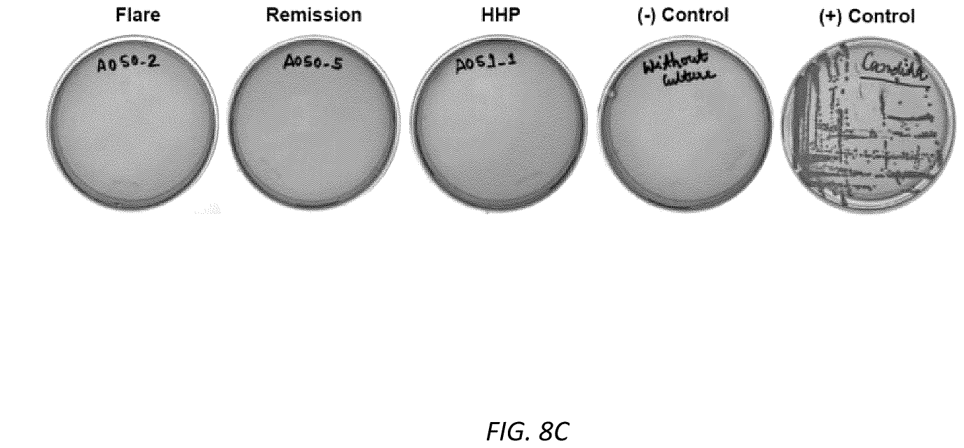

Analysis of the gut fungal microbiome by ITS2 sequencing demonstrated no significant differences in gut fungal composition between patients with Auto-brewery Syndrome and household partners (FIGS. 8A, 8B). Further, analysis by MaAsLin2 identified no significantly differentially abundant fungal genera between the different groups, and there was no fungal growth from stool samples from patients with Auto-brewery Syndrome or household partners (FIG. 8C). These data suggest that gut bacteria are important contributors to Auto-brewery Syndrome pathophysiology. However, since antifungal use increased in patients after an Auto-Brewery Syndrome diagnosis (Table 2), it is possible that intestinal fungal growth was suppressed by the time stool samples were collected. Therefore, the potential involvement of fungi in certain patients cannot be dismissed.

### Example 4. Functional Profiling Reveals Importance of Ethanol Fermentation Pathways

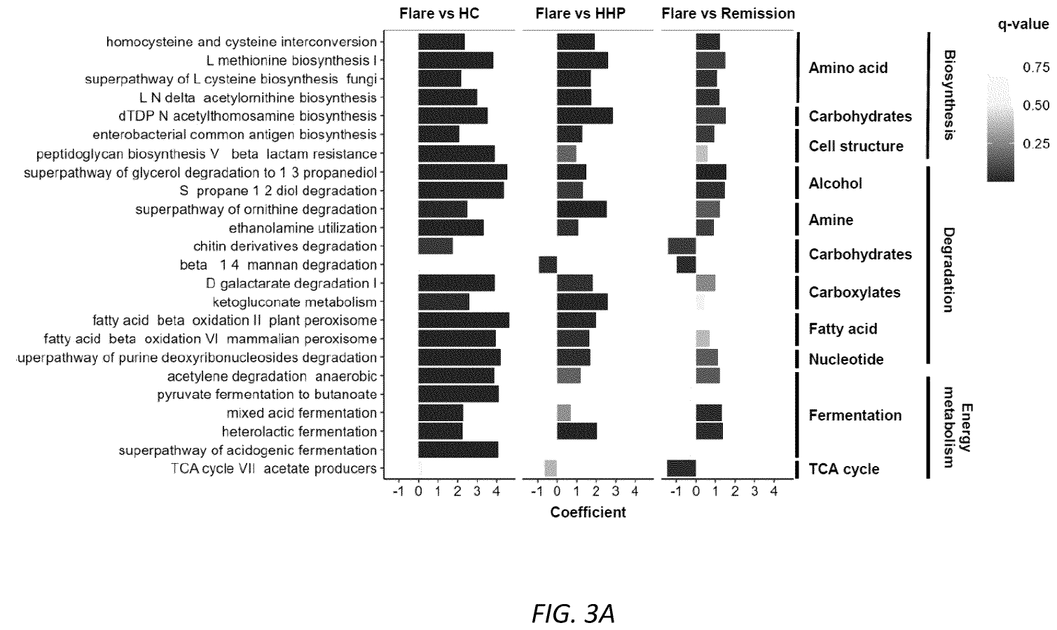

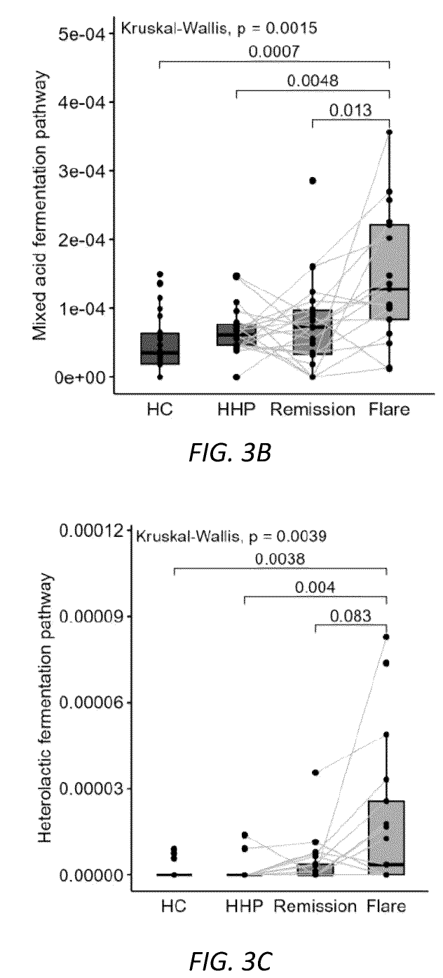

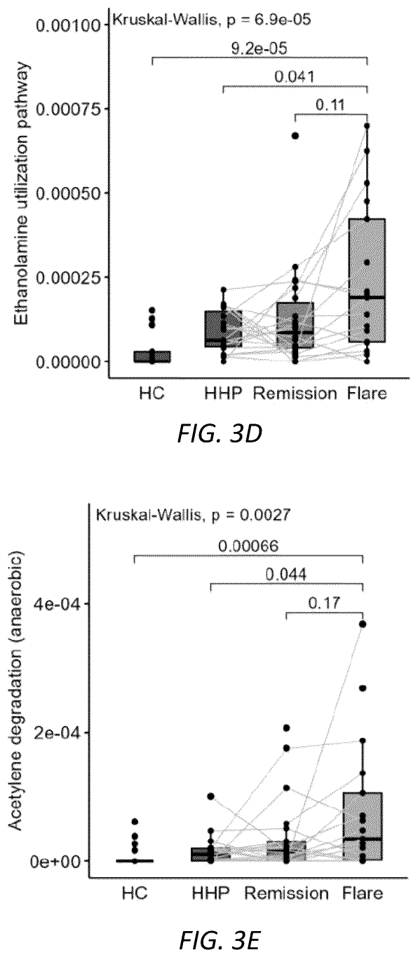

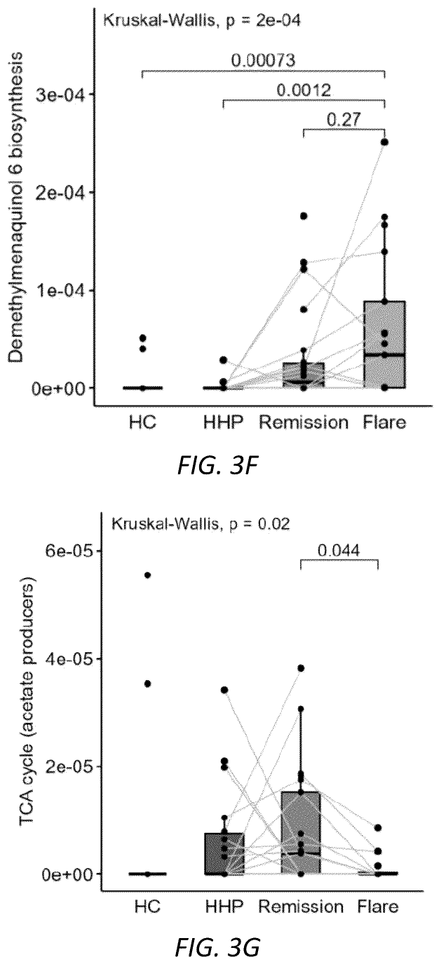

Using MAsLin2 to identify significant differences in metabolic pathway abundance between the different conditions while controlling for the random effects of each household, 18 metabolic pathways were significantly differentially abundant between “Flare” and “Remission” samples and 133 microbial pathways were significantly differentially abundant between “Flare” and household partner samples (q<0.05). FIG. 3a illustrates the differentially abundant microbial metabolic pathways that distinguish between disease states, categorized into three functional groups (biosynthesis, degradation, and energy metabolism) and their respective subgroups.

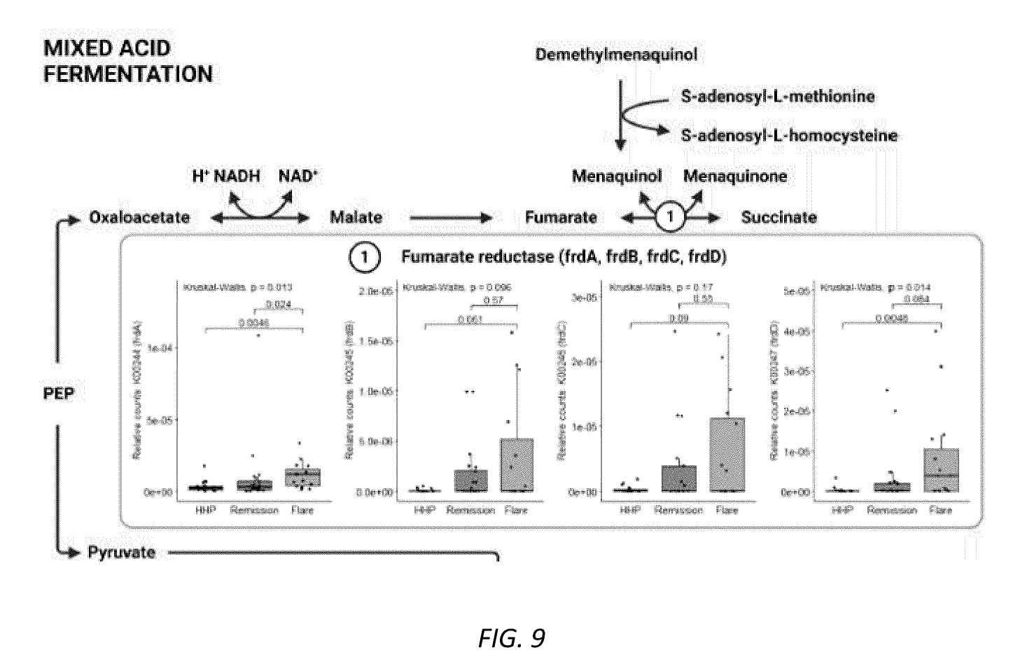

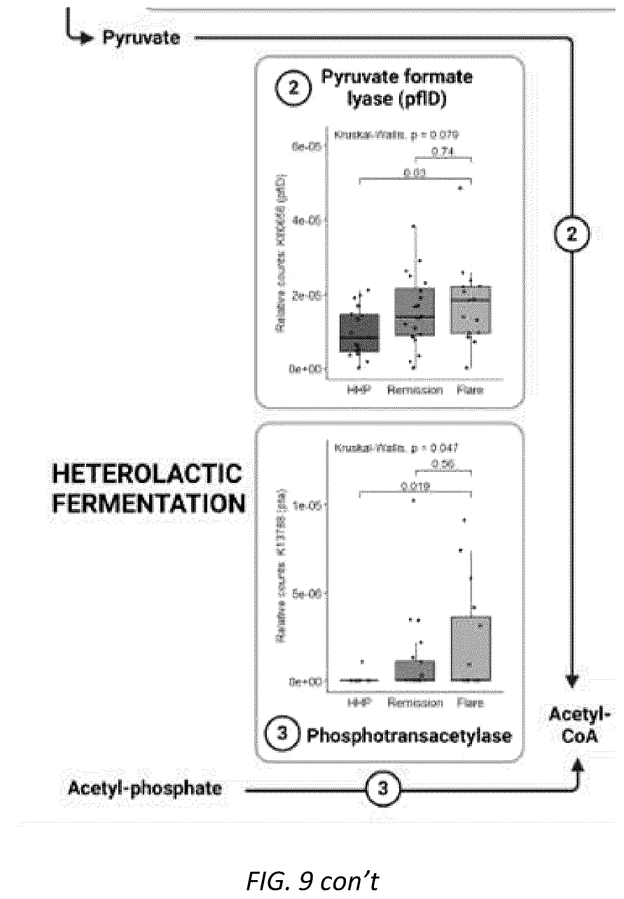

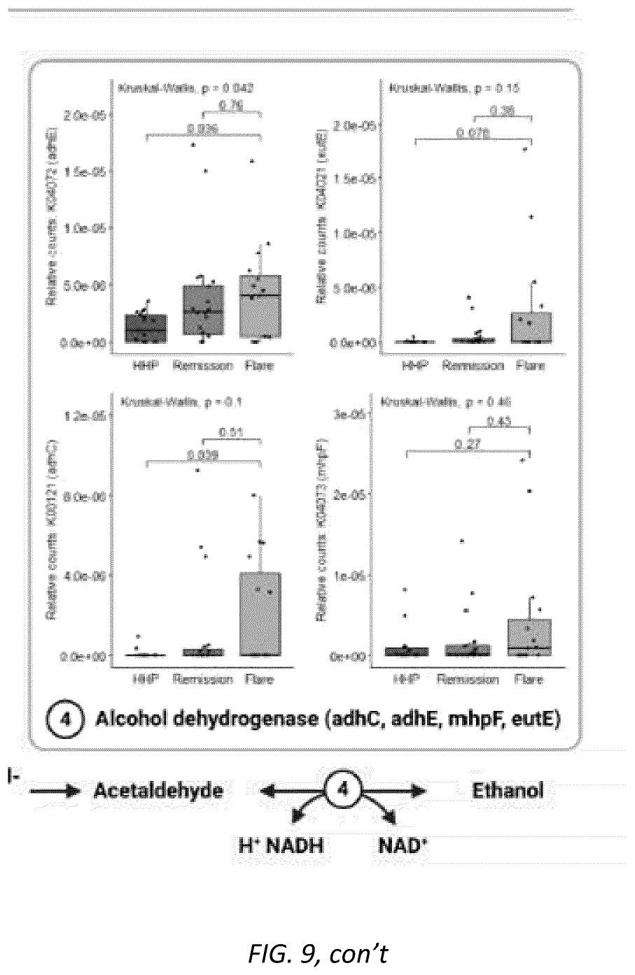

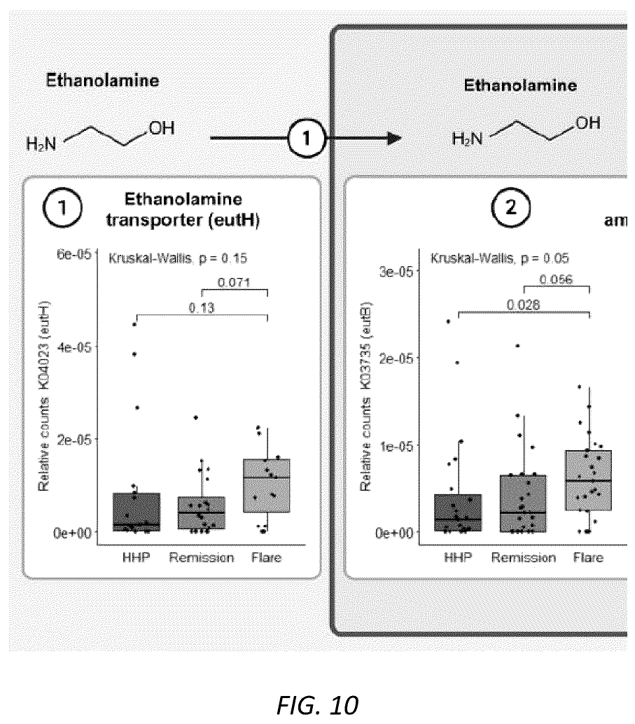

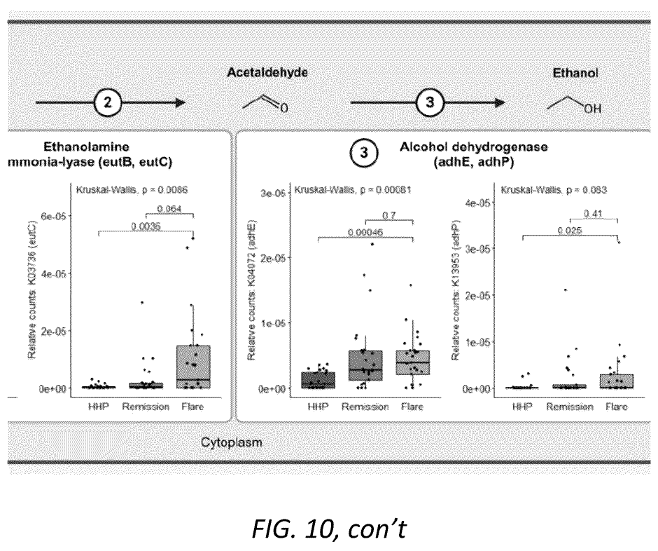

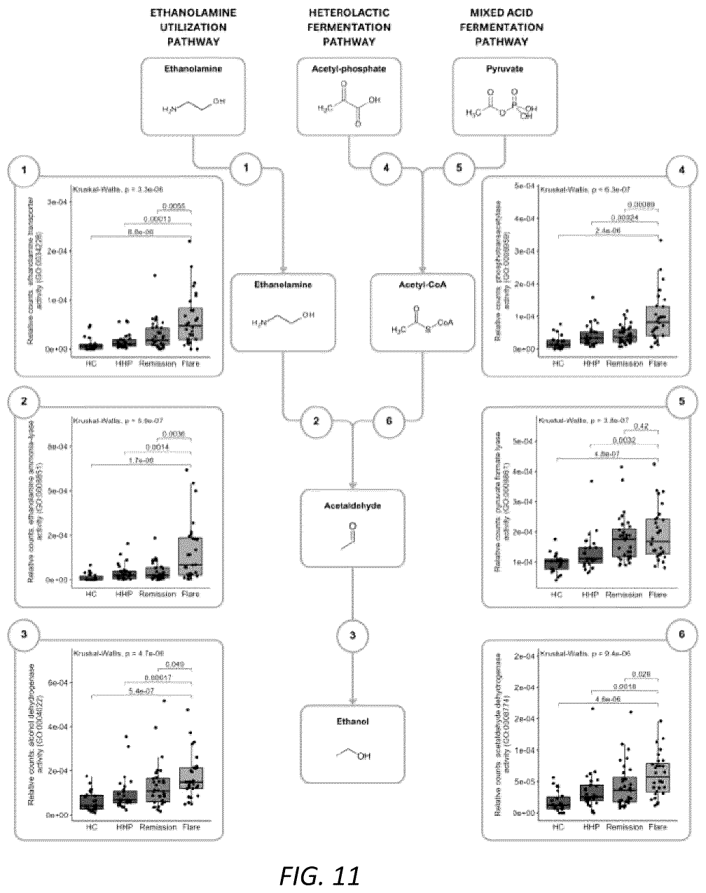

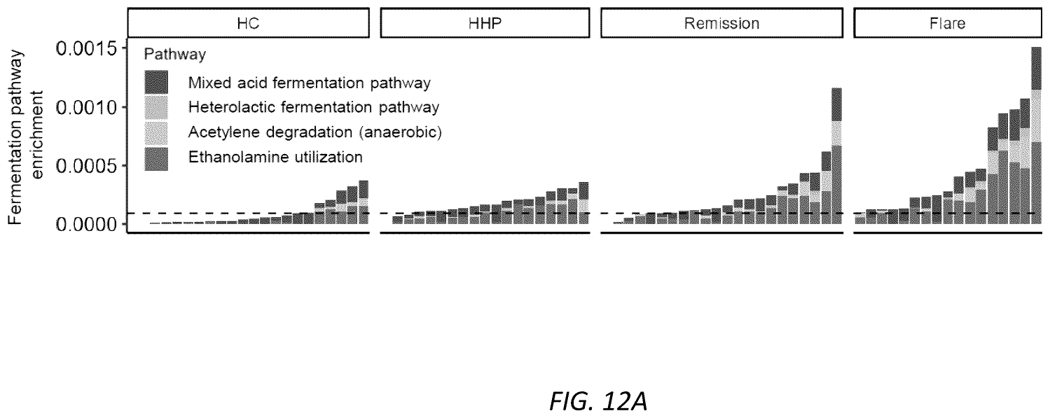

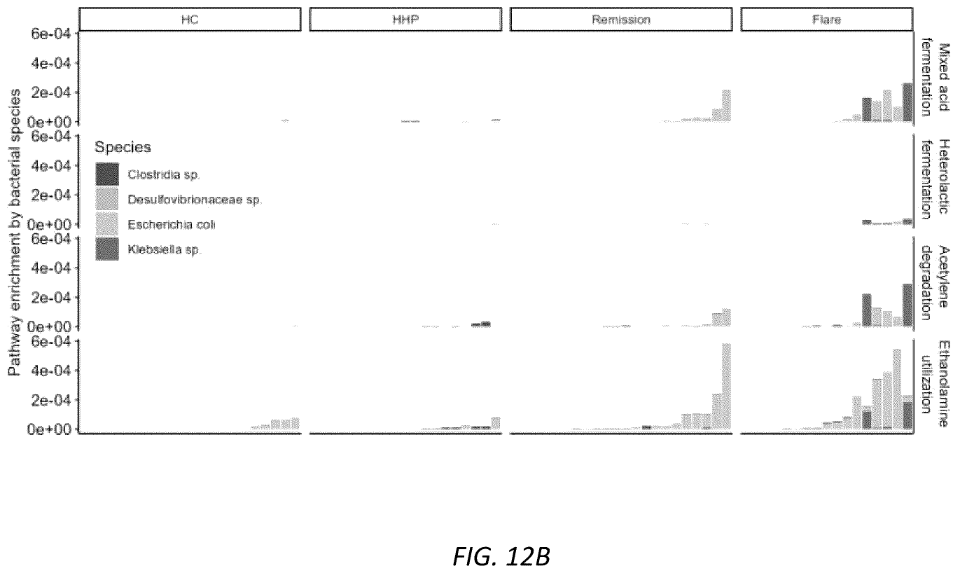

Two fermentation pathways commonly used by bacteria to produce ethanol were significantly more abundant in patients with clinically documented Auto-brewery Syndrome—the heterolactic fermentation pathway and the mixed acid fermentation pathway (FIGS. 3A-3C). In the heterolactic fermentation pathway, glucose is metabolized through the phosphoketolase pathway to acetyl phosphate, which is subsequently converted to ethanol through oxidation [24]. The mixed acid fermentation pathway allows for the conversion of glucose into ethanol and succinate [25]. N early every Kyoto Encyclopedia of Genes and Genomes (K EGG) orthology-annotated enzyme in these two pathways is significantly over-represented in the flare microbiome samples compared with the remission and household partner samples from within the same household (FIG. 9, FIG. 12A). The mixed acid fermentation and heterolactic fermentation pathway gene content increases in the patients with Auto-brewery Syndrome were largely attributed to two species within the Proteobacteria phylum, Escherichia coli and Klebsiella pneumoniae (FIG. 12B), with a minimal contribution was attributable to Clostridia and Desulfovibrionaceae species. Similarly, the ethanolamine utilization pathway, which allows for the transportation of ethanolamine into the cell and conversion to ethanol, was also increased in samples from patients in a flare compared with household partners and healthy controls (FIGS. 3A-3D, FIG. 10, FIG. 12A). The Gene Ontology (GO)-annotated molecular functions contributing to the ethanolamine utilization pathway, the heterolactic fermentation pathway, and the mixed acid fermentation pathway for ethanol production are also significantly enriched in samples from patients in a flare compared with remission, household partners, and healthy controls (FIG. 11). Additionally, the acetylene degradation pathway, which converts acetylene to ethanol, was also increased in samples from patients in a flare compared with household partners and healthy controls (FIGS. 3A, 3E). Several pathways important for menaquinol and demethylmenaquinol biosynthesis were also increased in samples from patients in a flare compared with household partners and healthy controls (FIG. 3F). Facultative anaerobes from the Gammaproteobacteria class such as E. coli utilize low-potential quinones such as menaquinone and demethylmenaquinone as electron acceptors mainly in anaerobic respiration [26], which is also the setting for alcohol fermentation. In contrast, the TCA cycle VII, used by acetate-producing bacteria to oxidize ethanol to acetate, was significantly under-represented in flare samples compared with remission samples (FIGS. 3A, 3G).

### Example 5. Gut Microbial Ethanol Production Causes Liver Injury In Vivo

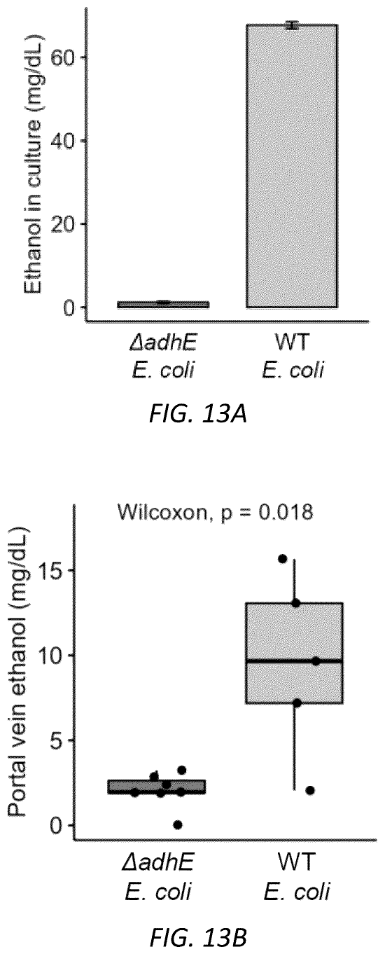

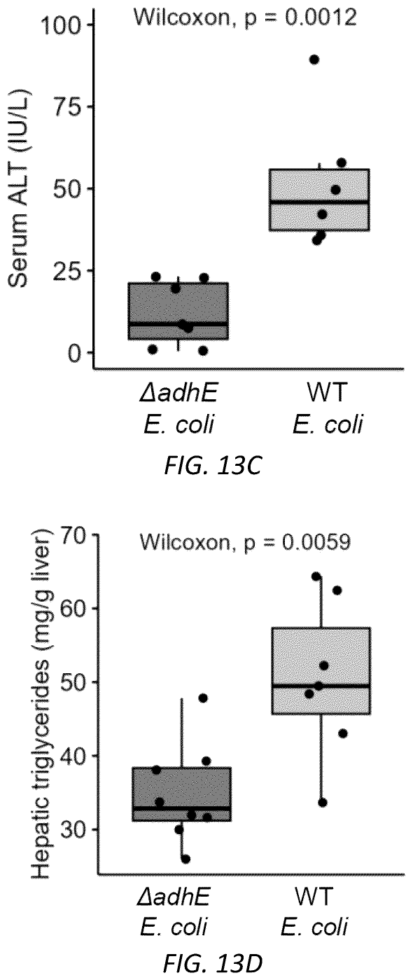

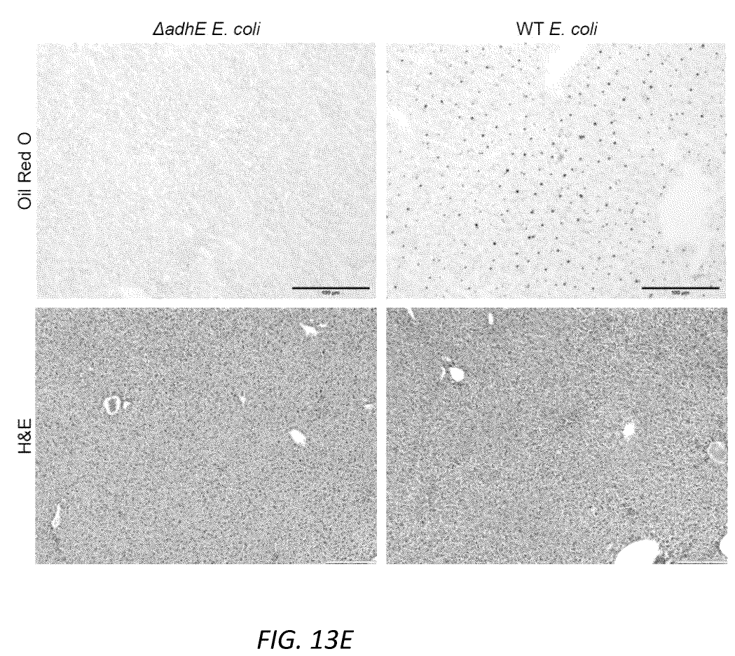

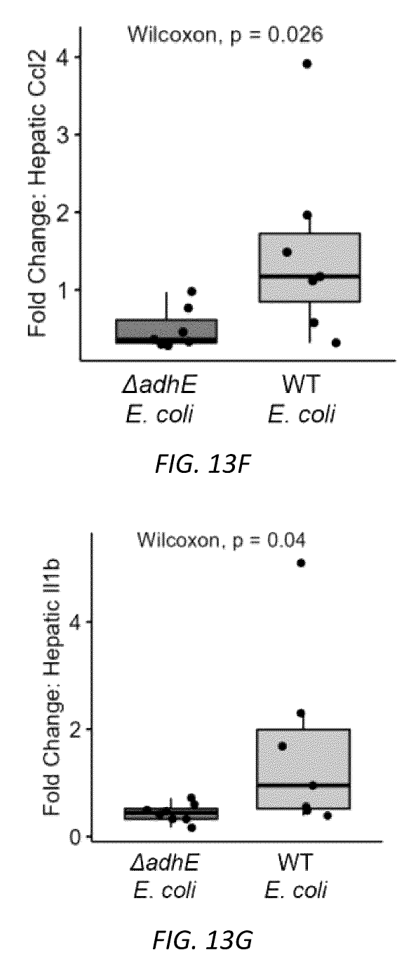

Acetaldehyde-alcohol dehydrogenase (adhE) enzymes, which are conserved across all bacterial kingdoms, facilitate conversion of acetyl-CoA to ethanol via an acetaldehyde intermediate during anaerobic ethanol fermentation [27]. Gut microbes in patients with ABS during a flare have much higher adhE-assigned gene counts (FIG. 9) and gene ontology-predicted alcohol dehydrogenase activity (FIG. 11). To highlight the importance of bacterial alcohol dehydrogenase activity as the cause of gut microbial ethanol production in vivo, germ-free mice were colonized with either wild-type (WT) E. coli or a genetically identical (isogenic) strain of E. coli with knock-out of the alcohol dehydrogenase gene (ΔadhE) and maintained them on a standard chow diet with 2% glucose water ad libitum for 10 weeks. The amount of food and water consumption between the two groups were comparable. Ethanol levels were undetectable in cultures of ΔadhE E. coli grown in LB media (FIG. 13A).

After 10 weeks, gnotobiotic mice colonized with wild-type E. coli demonstrated significantly more liver injury, as measured by serum ALT (FIG. 13C), and more hepatic steatosis, as evaluated by hepatic triglycerides (FIG. 13D) and Oil Red 0 staining on liver sections (FIG. 13E) as compared with mice colonized with ΔadhE E. coli. Hepatic mRNA expression of inflammatory cytokines C—C motif ligand 2 (Cc12) and Interleukin-1β (II1b) was also increased in wild-type E. coli-colonized mice as compared with ΔadhE E. coli-colonized mice. These results mirror the liver injury seen in patients with Auto-brewery Syndrome and highlight gut microbial ethanol production through the adhE pathway as an important contributor.

### Example 6. Gut Microbial Alterations and Endogenous Ethanol Production after Fecal Microbiota Transplantation Correlate with Symptoms

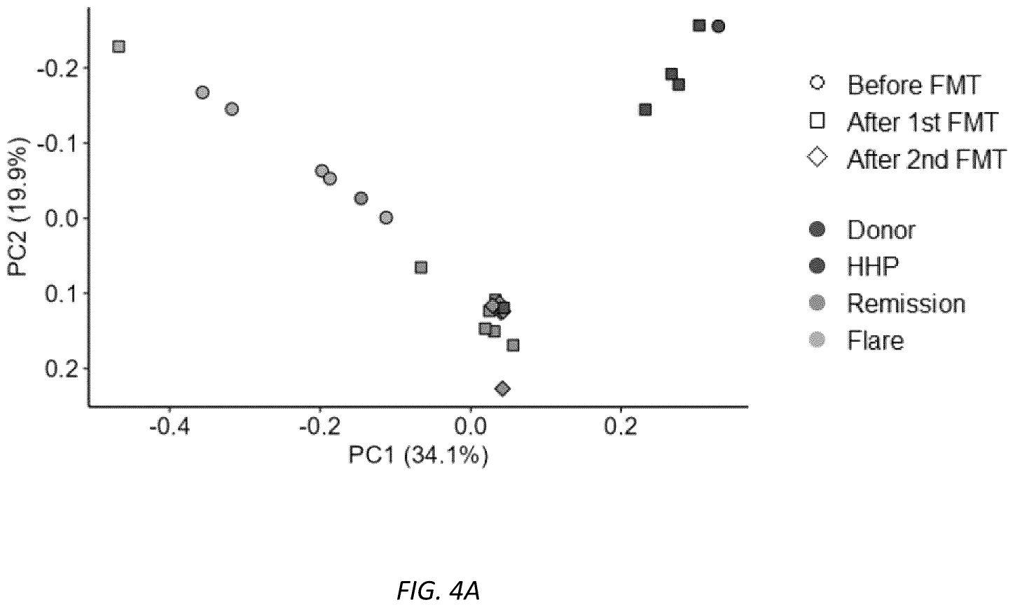

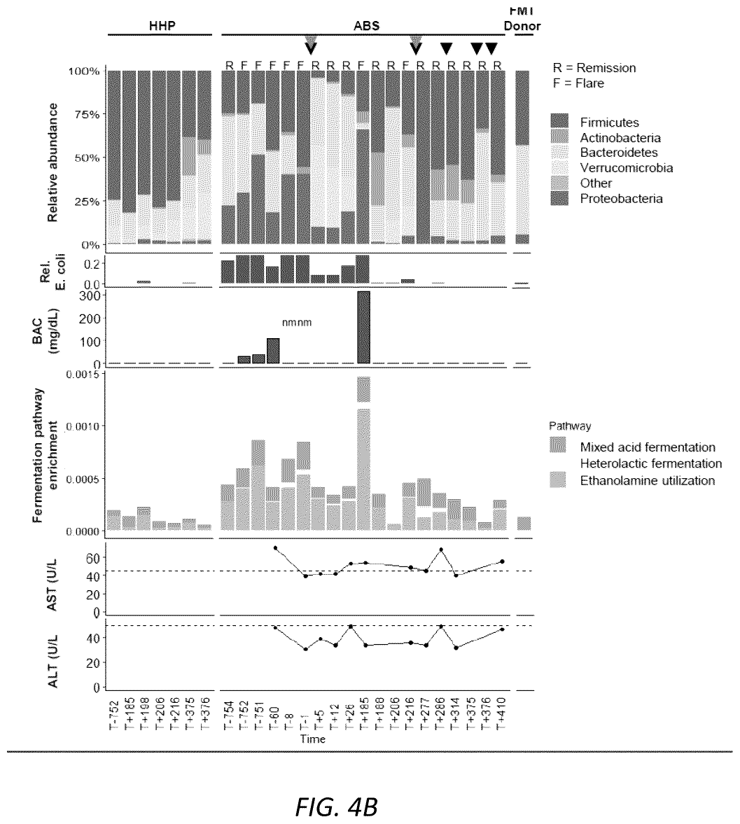

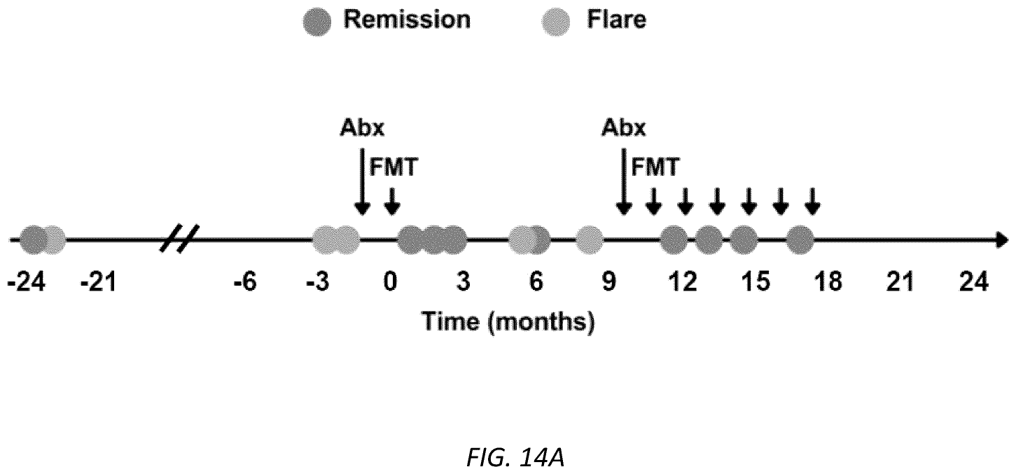

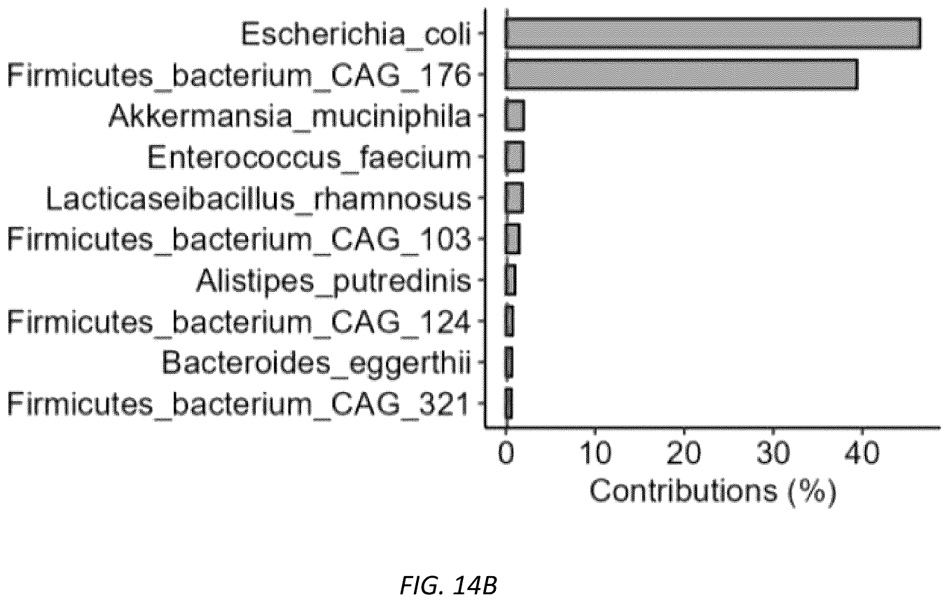

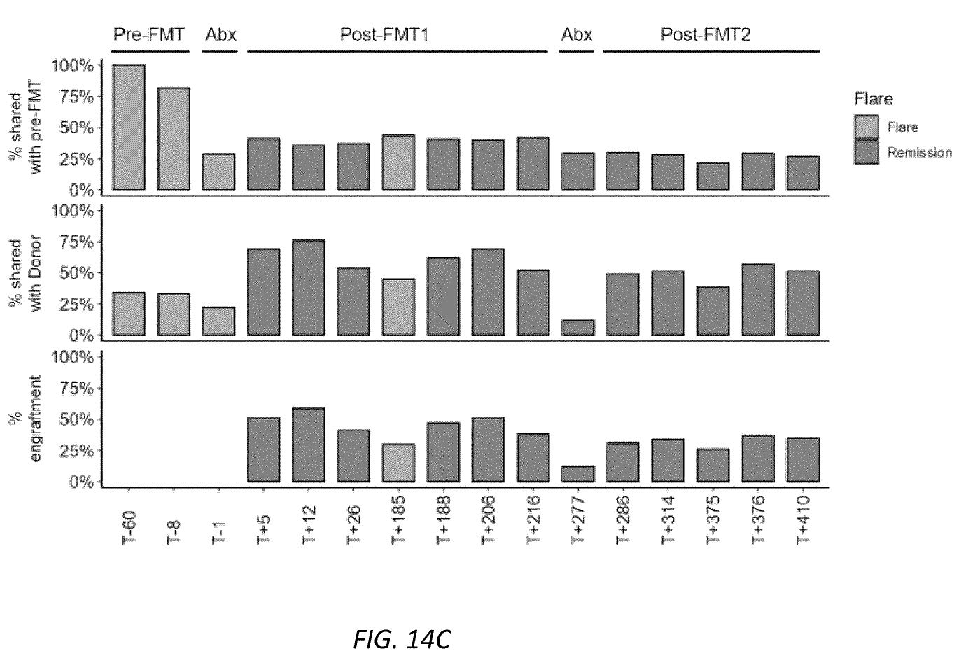

One subject A 20 with clinically documented Auto-brewery Syndrome was treated with fecal microbiota transplantation (FMT) twice (FIG. 14A) as described in the Methods section. After initial FMT, the patient experienced significant improvement in symptoms and no detectable blood alcohol levels for 3 months. The patient experienced greater wakefulness, less somnolent periods, and less belligerence, and family members noted that his pre-morbid behavior patterns had essentially returned. Unfortunately, his symptoms relapsed after this initial period of remission. Nine months after initial FMT, the patient received a second FMT, utilizing a more aggressive antibacterial pretreatment regimen, and including monthly maintenance dosing for six months. Additional complementary approaches were added because the patient was so severely affected and wished to do anything possible to avoid relapse. The patient remained asymptomatic/in remission for over 16 months after the second FMT, and advanced his diet to include more complex carbohydrates. Metagenomic analyses of the stool samples collected before, during, and after the first and second FMT correlate closely with the patient's symptoms (FIG. 4A). Notably, principal coordinate analysis demonstrated that after the first FMT, the composition of the patient's gut microbiota during remission clustered closely with the FMT donor gut microbiota, but the composition changed significantly during a flare episode. After the second FMT, the composition of the patient's gut microbiota remained closely clustered with the FMT donor gut microbiota. The bacterial species that contributed most to the first and second principal components were Escherichia coli, Firmicutes bacterium, Akkermansia muciniphila, and Enterococcus faecium (FIG. 14B). During flares of symptoms, the patient's gut microbiota contained an overabundance of Proteobacteria and an over-representation of the metabolic pathways associated with fermentation of alcohol (FIG. 4B). The patient's symptoms also correlated with the fraction of post-FMT species shared with pre-FMT (shared species between post-FMT and pre-FMT divided by the number of species profiled at post-FMT), the fraction of post-FMT species shared with the donor (shared species between post-FMT and donor divided by the number of species profiled at post-FMT), and percent engraftment as the number of shared species between post-FMT and the donor excluding the strains shared between pre-FMT and the donor samples (FIG. 14C) [28]. These metagenomic sequencing results were confirmed by single colony isolation of high ethanol-producing strains of Escherichia coli from this patient's stool samples.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. Painter, K., Cordell, B. J. & Sticco, K. L. Auto-Brewery Syndrome. (2024).
2. Bayoumy, A. B., Mulder, C. J. J., Mol, J. J. & Tushuizen, M. E. Gut fermentation syndrome: A systematic review of case reports. United European Gastroenterol J 9, 332-342 (2021).
3. Xue, G., et al. Three Klebsiella species as potential pathobionts generating endogenous ethanol in a clinical cohort of patients with auto-brewery syndrome: a case control study. EBioMedicine 91, 104560 (2023).
4. Dinis-Oliveira, R. J. The Auto-Brewery Syndrome: A Perfect Metabolic “Storm” with Clinical and Forensic Implications. J Clin Med 10(2021).
5. Malik, F., Wickremesinghe, P. & Saverimuttu, J. Case report and literature review of auto-brewery syndrome: probably an underdiagnosed medical condition. BMJ Open Gastroenterol 6, e000325 (2019).
6. Dahshan, A. & Donovan, K. Auto-brewery syndrome in a child with short gut syndrome: case report and review of the literature. J Pediatr Gastroenterol Nutr 33, 214-215 (2001).
7. Welch, B. T., Coelho Prabhu, N., Walkoff, L. & Trenkner, S. W. Auto-brewery Syndrome in the Setting of Long-standing Crohn's Disease: A Case Report and Review of the Literature. J Crohns Colitis 10, 1448-1450 (2016).
8. Yuan, J., et al. Fatty Liver Disease Caused by High-Alcohol-Producing Klebsiella pneumoniae. Cell Metab 30, 675-688 e677 (2019).
9. Youngster, I., et al. Oral, capsulized, frozen fecal microbiota transplantation for relapsing Clostridium difficile infection. JAMA 312, 1772-1778 (2014).
10. Riwes, M. M., et al. Feasibility of a dietary intervention to modify gut microbial metabolism in patients with hematopoietic stem cell transplantation. Nat Med 29, 2805-2813 (2023).
11. Ferraris, R. P., Yasharpour, S., Lloyd, K. C., Mirzayan, R. & Diamond, J. M. Luminal glucose concentrations in the gut under normal conditions. Am J Physiol 259, G822-837 (1990).
12. Marotz, C., et al. SARS-CoV-2 detection status associates with bacterial community composition in patients and the hospital environment. Microbiome 9, 132 (2021).
13. sergio.baranzini@ucsf.edu, i.C.E.a. & Consortium, i. Gut microbiome of multiple sclerosis patients and paired household healthy controls reveal associations with disease risk and course. Cell 185, 3467-3486.e3416 (2022).
14. Beghini, F., et al. Integrating taxonomic, functional, and strain-level profiling of diverse microbial communities with bioBakery 3. Elife 10(2021).
15. Blanco-Miguez, A., et al. Extending and improving metagenomic taxonomic profiling with uncharacterized species using MetaPhlAn 4. Nat Biotechnol 41, 1633-1644 (2023).
16. Mallick, H., et al. Multivariable association discovery in population-scale meta-omics studies. PLoS Comput Biol 17, e1009442 (2021).
17. Andrews, S.n.d.F.A.Q.C.T.f.H.T.S.D.
18. Bushnell, B.n.d.B.s.n.p.b.
19. Callahan, B. J., et al. DADA2: High-resolution sample inference from Illumina amplicon data. Nat Methods 13, 581-583 (2016).
20. Baba, T., et al. Construction of Escherichia coli K-12 in-frame, single-gene knockout mutants: the Keio collection. Mol Syst Biol 2, 2006.0008 (2006).
21. Thiele, C. & Hirschfeld, G. cutpointr: Improved Estimation and Validation of Optimal Cutpoints in R. Journal of Statistical Software 98, 1-27 (2021).
22. Meadows, V., Antonio, J.M., Ferraris, R.P. & Gao, N. Ruminococcus gnavus in the gut: driver, contributor, or innocent bystander in steatotic liver disease?FEBS J 292, 1252-1264 (2025).
23. Fusco, W., et al. Short-Chain Fatty-Acid-Producing Bacteria: Key Components of the Human Gut Microbiota. Nutrients 15(2023).
24. Nelson, M. E. & Werkman, C. H. Dissimilation of Glucose by Heterofermentative Lactic Acid Bacteria. J Bacteriol 30, 547-557 (1935).
25. Clark, D. P. The fermentation pathways of Escherichia coli. FEMS Microbiol Rev 5, 223-234 (1989).
26. Rendon, J., et al. Demethylmenaquinol is a substrate of Escherichia coli nitrate reductase A (NarGHI) and forms a stable semiquinone intermediate at the NarGHI quinol oxidation site. Biochim Biophys Acta 1847, 739-747 (2015).
27. Pony, P., Rapisarda, C., Terradot, L., Marza, E. & Fronzes, R. Filamentation of the bacterial bi-functional alcohol/aldehyde dehydrogenase AdhE is essential for substrate channeling and enzymatic regulation. Nat Commun 11, 1426 (2020).
28. Ianiro, G., et al. Variability of strain engraftment and predictability of microbiome composition after fecal microbiota transplantation across different diseases. Nat Med 28, 1913-1923 (2022).
29. Kurosu, M. & Begari, E. Vitamin K2 in electron transport system: are enzymes involved in vitamin K2 biosynthesis promising drug targets?Molecules 15, 1531-1553 (2010).
30. Whistance, G. R. & Threlfall, D. R. Effect of anaerobiosis on the concentrations of demethylmenaquinone, menaquinone and ubiquinone in Escherichia freundii, Proteus mirabilis and Aeromonas punctata. Biochem J 108, 505-507 (1968).
31. Peterson, J. D. & Ingram, L. O. Anaerobic respiration in engineered Escherichia coli with an internal electron acceptor to produce fuel ethanol. Ann N Y Acad Sci 1125, 363-372 (2008).
32. Vandekerckhove, E., Janssens, F., Tate, D. & De Looze, D. Treatment of Gut Fermentation Syndrome With Fecal Microbiota Transplantation. Ann Intern Med 173, 855 (2020).
33. Gan, L., et al. Bacteriophage targeting microbiota alleviates non-alcoholic fatty liver disease induced by high alcohol-producing Klebsiella pneumoniae. Nat Commun 14, 3215 (2023).
34. Meijnikman, A. S., et al. Microbiome-derived ethanol in nonalcoholic fatty liver disease. Nat Med 28, 2100-2106 (2022).
35. Hafez, E. M., Hamad, M. A., Fouad, M. & Abdel-Lateff, A. Auto-brewery syndrome: Ethanol pseudo-toxicity in diabetic and hepatic patients. Hum Exp Toxicol 36, 445-450 (2017).
36. Zhu, L., et al. Characterization of gut microbiomes in nonalcoholic steatohepatitis (NASH) patients: a connection between endogenous alcohol and NASH. Hepatology 57, 601-609 (2013).

What is claimed is:

1. A method for treating a condition associated with gut alcohol production in a subject, the method comprising:
(a) analyzing a fecal sample obtained from a subject, wherein analyzing the fecal sample comprises culturing or having cultured the fecal sample and performing or having performed metagenomic sequencing of the fecal sample;
(b) determining that the subject has a gut alcohol production condition; and
(c) administering to the subject an effective amount of a treatment comprising a probiotic with optional resistant starch, fecal microbiota transplantation (FMT), an antimicrobial agent, a microbial enzyme inhibitor, or a combination thereof.

2. The method of claim 1, wherein the fecal sample is obtained from the subject if the subject is experiencing or has experienced symptoms of a condition associated with gut alcohol production.

3. The method of claim 2, wherein the fecal sample is obtained from the subject if the subject has experienced three or more episodes of symptoms of a condition associated with gut alcohol production within the last year.

4. The method of claim 1, wherein the condition associated with gut alcohol production comprises Auto Brewery Syndrome (ABS), steatotic liver disease, or alcohol use disorder.

5. The method of claim 4, wherein the steatotic liver diseases comprise metabolic dysfunction-associated steatotic liver disease (MASLD), metabolic dysfunction and alcohol-associated liver disease (MetALD), or alcohol-associated liver disease (ALD).

6. The method of claim 1, wherein culturing or having cultured the fecal sample comprises culturing the fecal sample in a bioreactor.

7. The method of claim 1, wherein performing or having performed metagenomic sequencing of the fecal sample comprises shotgun metagenomic sequencing of the fecal sample.

8. The method of claim 1, wherein performing or having performed metagenomic sequencing of the fecal sample is not and/or does not include 16S rRNA sequencing.

9. The method of claim 1, wherein determining the subject has a gut alcohol production condition comprises identifying the fecal sample as meeting one or more of the following conditions:
(a) the amount of ethanol produced by the fecal sample is higher than 8.21 mg/dL in bioreactor culture;
(b) the abundance of Ruminococcus gnavus, Escherichia coli, or Klebsiella pneumoniae is higher in the fecal sample obtained from the subject as compared to a reference fecal sample;
(c) enzymes in the heterolactic fermentation pathway, the mixed acid fermentation pathway, the ethanolamine utilization pathway, and/or acetylene degradation pathway are over-represented in the fecal sample compared to a reference fecal sample; and
(d) genes associated with one or more of the enzymes identified in (c) are over-represented in the fecal sample compared to a reference fecal sample.

10. The method of claim 9, wherein the fecal sample meets two or more of the conditions.

11. The method of claim 9, wherein the amount of ethanol produced by the fecal sample is determined using anaerobic bioreactor culture and high-performance liquid chromatography (HPLC).

12. The method of claim 9, wherein the enzymes comprise one or more of fumarate reductase, pyruvate formate lyase, phosphotransacetylase, acetaldehyde dehydrogenase, alcohol dehydrogenase (ADH), ethanolamine transporter, and ethanolamine ammonia-lyase.

13. The method of claim 12, wherein the ADH comprises E. coli ADH.

14. The method of claim 1, wherein the antimicrobial agent comprises an antibacterial agent and/or an antifungal agent.

15. The method of claim 1, wherein the antibacterial agent comprises chloramphenicol.

16. The method of claim 1, wherein the microbial enzyme inhibitor comprises thiabendazole, cambendazole, fenbendazole, oxfendazole, methacrylate, acryalate, metadoxine, disulfiram, fomepizole, or combinations thereof.

17. A method of analyzing a fecal sample, comprising:
(a) measuring the amount of ethanol produced by the fecal sample; and
(b) measuring the relative abundance of Ruminococcus gnavus, Escherichia coli, or Klebsiella pneumoniae in the fecal sample compared to that in a reference sample, and/or measuring the expression of genes associated with the heterolactic fermentation pathway, the mixed acid fermentation pathway, the ethanolamine utilization pathway, and/or acetylene degradation pathway,
(c) thereby analyzing a fecal sample.

18. The method of claim 17, wherein measuring the amount of ethanol comprises culturing the fecal sample in a bioreactor and determining ethanol production of the cultured fecal sample by chromatography, spectrophotometry, or enzymatic assay.

19. The method of claim 18, wherein the chromatography is gas chromatography (GC) or high performance liquid chromatography (HPLC).

20. The method of claim 18, wherein the spectrophotometry comprises dichromate oxidation, UV-vis spectrophotometry, near-infrared (NIR) spectrophotometry, or probe-based spectrophotometry.

21. The method of claim 18, wherein the enzymatic assay comprises ADH assay, or fluorometric assays.

22. The method of claim 17, wherein measuring the expression of genes comprises shotgun metagenomic sequencing.
